SEQUENCE LISTING (30 sequences)

SEQ ID NO: 1 - DNA, 27 bp, Homo sapiens
aaaacaagatttacctctattgttgga

SEQ ID NO: 2 - DNA, 20 bp, Homo sapiens
aggcctgctgaaaatgactg

SEQ ID NO: 3 - DNA, 22 bp, Homo sapiens
gttaaaattcccgtcgctatca

SEQ ID NO: 4 - DNA, 17 bp, Homo sapiens
gacccccacacagcaaa

SEQ ID NO: 5 - DNA, 23 bp, Homo sapiens
aagttaaaattcccgtcgctatc

SEQ ID NO: 6 - DNA, 21 bp, Homo sapiens
gcagcatgtcaagatcacaga

SEQ ID NO: 7 - DNA, 20 bp, Homo sapiens
tgcctccttctgcatggtat

SEQ ID NO: 8 - DNA, 19 bp, Homo sapiens
aaccagccctgtcgtctct

SEQ ID NO: 9 - DNA, 20 bp, Homo sapiens
caagcagtcacagcacatga

SEQ ID NO: 10 - DNA, 18 bp, Homo sapiens
ctgagcagcgctcatggt

SEQ ID NO: 11 - DNA, 20 bp, Homo sapiens
gtgcagctgtgggttgattc

SEQ ID NO: 12 - DNA, 17 bp, Homo sapiens
gggggtgtggaatcaac

SEQ ID NO: 13 - DNA, 20 bp, Homo sapiens
acttgtgccctgactttcaa

SEQ ID NO: 14 - DNA, 20 bp, Homo sapiens
cagttgcaaaccagacctca

SEQ ID NO: 15 - DNA, 21 bp, Homo sapiens
ggcctctgattcctcactgat

SEQ ID NO: 16 - DNA, 20 bp, Homo sapiens
ggctcctgacctggagtctt

SEQ ID NO: 17 - DNA, 20 bp, Homo sapiens
cttgggcctgtgttatctcc

SEQ ID NO: 18 - DNA, 20 bp, Homo sapiens
tcttgcggagattctcttcc

SEQ ID NO: 19 - DNA, 21 bp, Homo sapiens
gcctcttgcttctcttttcct

SEQ ID NO: 20 - DNA, 20 bp, Homo sapiens
gcttcttgtcctgcttgctt

SEQ ID NO: 21 - DNA, 20 bp, Homo sapiens
ctactgggacggaacagctt

SEQ ID NO: 22 - DNA, 20 bp, Homo sapiens
ggtcctgcaccagtaatatg

SEQ ID NO: 23 - DNA, 20 bp, Homo sapiens
gcctgctgaaaatgactgaa

SEQ ID NO: 24 - DNA, 21 bp, Homo sapiens
agaatggtcctgcaccagtaa

SEQ ID NO: 25 - DNA, 20 bp, Homo sapiens
aaggcctgctgaaaatgact

SEQ ID NO: 26 - DNA, 22 bp, Homo sapiens
ttgttggatcatattcgtccac

SEQ ID NO: 27 - DNA, 20 bp, Homo sapiens
gctggtaacatccacccaga

SEQ ID NO: 28 - DNA, 20 bp, Homo sapiens
ccacacagcaaagcagaaac

SEQ ID NO: 29 - DNA, 20 bp, Homo sapiens
ttcccatgatgatctgtccc

SEQ ID NO: 30 - DNA, 20 bp, Homo sapiens
tctttctcttccgcacccag

(19) United States
(12) Patent Application Publication
(10) Pub. No.: US 2019/0382848 A1
(43) Pub. Date: Dec. 19, 2019

(54) Primers, Methods and Kits for Diagnosing and Predicting Therapy Response of Cancers by Cold-PCR Based Amplification of Mutation-Rich Regions of KRAS, EGFR and P53

(71) Applicant: IMPERIAL COLLEGE INNOVATIONS LIMITED, London (GB)

(72) Inventors: Eric Kian Saik Lim, London (GB); Maxim Borisovich Freydin, London (GB)

(21) Appl. No.: 16/464,098

(22) PCT Filed: Nov. 24, 2017
(86) PCT No.: PCT/GB2017/053539

§ 371 Date: May 24, 2019

(30) Foreign Application Priority Data
Nov. 25, 2016 (GB) 1619945.7

(51) Int. Cl. C12Q 1/6886; C12Q 1/686; C12Q 1/6858

(52) U.S. Cl. CPC C12Q 1/6886; C12Q 2600/156; C12Q 1/6858; C12Q 1/686

(57) ABSTRACT

Nucleic acids primers for use in the detection of mutations in KRAS, EGFR and P53 associated with cancer, and in particular provides nucleic acids and methods employing reaction conditions suitable for use in COLD-PCR and high resolution melting HRM analysis of circulating tumour DNA, particularly from lung and colon cancers. The invention further relates to a combination of KRAS and APC mutations in diagnosing cancer.

### FIELD OF THE INVENTION

The invention relates to the field of cancer diagnostics and treatment stratification.

It is well known that the mutations within tumours are very heterogeneous. For example one lung cancer tumour may have a KRAS mutation, whilst a tumour from another patient may have wild type KRAS. Similarly, the mutations within a particular gene are also heterogeneous and can cause a range of effects, for example a patient may have a mutation in the p53 tumour suppressor gene which has little effect on disease progression, whilst a patient with a different mutation in the p53 gene may have a very different prognosis.

Tumours are heterogeneous both between tumours, and also within an individual tumour. The heterogeneity of tumours, in particular of advanced cancers plays an important role in the resistance to therapy.

With the advent of personalised medicine, the ability to detect the presence of mutations in particular genes within a tumour, and also the type of mutation, allows therapy to be tailored to best target the particular tumour. For example, it is considered that tumours harbouring a mutation in the EGFR gene will benefit from anti-EGFR antibody therapy, whilst tumours lacking a mutation in this gene will not respond to the therapy.

Current techniques may be able to identify the presence of some mutations, but identification of, for example, low frequency mutations has been neglected leading to some mutations that just cannot possibly be detected with current techniques.

In addition, most current techniques are carried out on tissue samples or biopsies of the tumour (such as the Roche Cobas test), the taking of which is often an invasive procedure. There is therefore a need for a simple test that can be carried out on an easily-obtained sample of tumour nucleic acid, that can detect all possible known mutations in a given gene or locus.

In particular, with the knowledge of how each individual mutation within a gene influences tumour progression and response to various treatments, the ability to actually identify the particular mutation, rather than simply the presence of a mutation, will greatly enhance the targeting of a suitable therapy to a given tumour.

Lung cancer is the second most common cancer in the UK. In 2011 there were 46,463 new cases in the UK (Cancer Research UK). Of lung cancers, 72% are non-small cell lung cancer, of which 16.6% have EGFR-TK mutations and approximately 50% have a TP53 mutation (i.e. a mutation in the gene that encodes the p53 protein). 40% of lung cancers are adenocarcinomas, of which 30% carry a KRAS mutation. In one study (Yamaguchi et al 2012) out of 77 of patients that were tested, 27% had EGFR mutations, 1% had KRAS mutations, 36% had p53 mutations and 10% had both EGFR and p53 mutations.

Such mutations are also common in other cancers, for example in colorectal cancers (of which there were 41,581 new cases in the UK in 2011) 40-50% have mutations in TP53 (p53), 30-50% have mutations in the KRAS locus, and 25-82% have a mutation in the EGFR gene (this is dependent on the location of the tumour).

Lung cancer costs the UK economy approximately £2.4 billion, and bowel cancer £1.6 billion. Each lung cancer patient costs the UK healthcare system £9,071 annually, compared to £2,756 for bowel cancer, £1,584 for prostate cancer and £1,076 for breast cancer. The average healthcare spend on each cancer patient in the UK is £2,776 (luengo-Fernandez et al 2013).

The cost of a standard bronchoscopy and mediastinoscopy, for the taking of a biopsy of lung tissue, is approximately £4,000. The cost of an endobronchial ultrasound (EBUS) with transbronchial needle aspiration (TBNA) is approximately £1365 (NICE 2011).

Thus mutations in the KRAS, p53 and EGFR loci are important diagnostic, prognostic, and treatment stratification markers.

The KRAS proto-oncogene is often mutated in cancers such as colorectal cancer and lung cancer as described above, and is most often associated with smoking related lung cancer. The mutations most commonly occur in codons 12 and 13, as well as codon 61. In the majority of cases, these mutations are missense mutations which introduce an amino acid substitution at position 12, 13, or 61. The result of these mutations is constitutive activation of KRAS signalling pathways.

| | | | | | Capable of |
|---|---|---|---|---|---|
| | | | Frequency among | Frequency | being |
| | | | KRAS- | among KRAS- | identified by |
| | Nucleotide | Amino acid | mutated colorectal | mutated lung | Roche |
| Codon | mutation | substitution | cancers | adenocarcinoma | Cobas test |
| 12 | 34: G-T | G-C | 7.9% | 42% | YES |
| 12 | 34: G-C | G-R | 1.2-1.4% | 2% | YES |
| 12 | 34: G-A | G-S | 4.9-5.7% | 5% | YES |
| 12 | 35: G-C | G-A | 6.2-6.6% | 7% | YES |
| 12 | 35: G-A | G-D | 33.5-34.4% | 17% | YES |
| 12 | 35: G-T | G-V | 21.9-24.4% | 15-25% | YES |
| 13 | 37: G-T | G-C | <1% | 3% | NO |
| 13 | 37: G-C | G-R | <1% | <1% | NO |
| 13 | 37: G-A | G-S | <1% | <1% | NO |
| 13 | 38: G-C | G-A | <1% | <1% | NO |
| 13 | 38: G-A | G-D | 18.9-19.2% | 2% | YES |

Data compiled from www.mycancergenome.org http://www.mycancergenome.org/content/disease/colorectal-cancer/kras/35/ Lovly, C., L. Horn, W. Pao. 2012. KRAS Mutations in Non-Small Cell Lung Cancer (NSCLC). My Cancer Genome http://www.mycancergenome.org/content/disease/lung-cancer/kras/(Updated July 31).

EGFR is also commonly mutated in lung and colorectal cancers, in particular exon 19 can harbour in-frame insertions and deletions, whilst exon 21 can have 2 missense mutations as shown in the table below:

| | | | | Capable of |
|---|---|---|---|---|
| | | | | being |
| | | Frequency of | Frequency in | identified by |
| | Nucleotide | EGFR mutations | EGFR mutated | Roche |
| Codon | mutation | among NSCLC | NSCLC | Cobas test |
| Exon 19 | Deletion | ~10% in the USA | 48% | YES |
| | | ~35% in Asia | | |
| Exon 19 | Insertion | ~10% in the USA | 1% | YES |
| | | ~35% in Asia | | |
| Exon 21 | L858R | ~10% in the USA | 43% | YES |
| | | ~35% in Asia | | |
| Exon 21 | L861Q | ~10% in the USA | 2% | YES |
| | | ~35% in Asia | | |

Data compiled from www.mycancergenome.org

http://www.mycancemenome.org/content/disease/colorectal-cancer/kras/35/ Lovly, C., L. Horn, W. Pao. 2012. KRAS Mutations in Non-Small Cell Lung Cancer (NSCLC). My Cancer Genome http://www.mycancergenome.org/content/disease/lung-cancer/kras/(Updated July 31).

Approximately 15-25% of patients with lung adenocarcinoma have tumor associated KRAS mutations. KRAS mutations are uncommon in lung squamous cell carcinoma (Brose et al. 2002). In the majority of cases, these mutations are missense mutations which introduce an amino acid substitution at position 12,13, or 61. The result of these mutations is constitutive activation of KRAS signaling pathways.

In the vast majority of cases, KRAS mutations are found in tumors wild type for EGFR or ALK; in other words, they are non-overlapping with other oncogenic mutations found in NSCLC. Therefore, KRAS mutation defines a distinct molecular subset of the disease. KRAS mutations are found in tumors from both former/current smokers and never smokers. They are rarer in never smokers and are less common in East Asian vs. US/European patients (Riely et al. 2008; Sun et al. 2010).

The role of KRAS as either a prognostic or predictive factor in NSCLC is unknown at this time. Very few prospective randomized trials have been completed using KRAS as a biomarker to stratify therapeutic options in the metastatic setting. Unlike in colon cancer, KRAS mutations have not yet been shown in NSCLC to be negative predictors of benefit to anti-EGFR antibodies. However, KRAS mutations are negative predictors of radiographic response to the EGFR tyrosine kinase inhibitors, erlotinib and gefitinib [for review, see (Riely and Ladanyi 2008; Riely, Marks, and Pao 2009)]. Currently, there are no direct anti-KRAS therapies available.

P53 is also often found mutated in cancers. Most p53 mutations are detected by DNA sequencing. However, it is known that single missense mutations can have a large spectrum from rather mild to very severe functional effects.

The large spectrum of cancer phenotypes due to mutations in the TP53 gene is also supported by the fact that different isoforms of p53 proteins have different cellular mechanisms for prevention against cancer. Mutations in TP53 can give rise to different isoforms, preventing their overall functionality in different cellular mechanisms and thereby extending the cancer phenotype from mild to severe. Recent studies show that p53 isoforms are differentially expressed in different human tissues, and the loss-of-function or gain-of-function mutations within the isoforms can cause tissue-specific cancer or provides cancer stem cell potential in different tissues (Bullock A N, Henckel J, DeDecker B S, Johnson C M, Nikolova P V, Proctor M R et al. (1997). “Thermodynamic stability of wild-type and mutant p53 core domain”. Proc. Natl. Acad. Sci. U.S.A. 94 (26): 14338-42. doi:10.1073/pnas.94.26.14338. PMC 24967. PMID 9405613; Burdon Jean-Christophe, Fernandes Kenneth, Murray-Zmijewski Fiona, Liu Geng, Diot Alexandra, Xiordimas Dimitris P., Saville Mark K., Lane David P. (September 2005). “p53 isoforms can regulate p53 functional activity”. Genes & Development 19 (18): 2122-37. doi:10.1101/gad.1339905. PMID 1221884; Khoury Marie P, Bourdon J C (April 2011). “p53 Isoform—An Intracellular Microprocessor?”. Genes Cancer. 4 (2): 453-465. doi:10.1177/1947601911408893. PMID 3135639; Avery-Kiejda K A, Morten B, Wong-Brown M W, MatheA, Scott R J (March 2014). “The relative mRNA expression of p53 isoforms is associated with clinical features andoutcome.”. Carcinogenesis 35 (3): 586-596. doi:10.1093/carcin/bgt411. PMID 24336193; Arsic Nikola, Gadea Gilles, LAgerqvist E. Louise, Busson Muriel, Cahuzac Nathalie, Brock Carsten, Hollande Frederic, Gire Veronique, Pannequin Julie, Roux Pierre (April 2015). “The p53 isoform of Δ133p53(3 Promotes Cancer Stem CellPotential”. Stem Cell Reports 4 (4): 531-540. doi:10.1016/j.stemcr.2015.02.001).

Tumours harbouring KRAS mutations are generally wild-type for pro-cancer mutations in the EGFR gene. Tumours with wild-type EGFR are notoriously resistant to EGFR-targeted therapies, such as monoclonal antibody therapy. Therefore activating mutations in KRAS are recognized as a strong predictor of resistance to EGFR-targeted mAbs. Routine testing of all patients with colorectal cancer for KRAS mutations is now recommended; only those harbouring wild-type (WT) KRAS should be candidates for such therapies, thus improving outcomes, and minimizing unnecessary toxicity and cost.

The relative effects of each different mutation in the KRAS gene, for example those detailed in the above table, are currently unknown. Following further research it may be possible to specifically target particular KRAS mutants with particular drugs. Therefore a routine technique that will allow the identification of any one of the 12 mutations detailed above and other less frequent mutations is desired, to enable the characterisation of a particular tumour, and therefore ultimately to tailor the therapeutic agent.

Additionally, a routine and reliable test to identify the presence of any one or more of the above mutations will also help discriminate tumours between those which are likely to also have an EGFR mutation (i.e. tumours with none of the above KRAS mutants) and are therefore suitable for anti-EGFR therapy, and those unlikely to have an EGFR mutation and that are thus unlikely to benefit from anti-EGFR therapy.

The EGFR gene is also commonly mutated in cancers. It is a prognostic and predictive gene mutation. The lung cancers in patients with selected EGFR mutations (e.g. Ex 19 del and L858R) are known to respond well to EGFR tyrosine kinase inhibitors.

Therefore, a simple, reliable, sensitive assay that can determine the presence of mutation in specific regions of the gene is highly desired.

Standard tests, such as the Roche™ Cobas™ tests for KRAS mutation are performed on formalin-fixed, paraffin-embedded tissue, and therefore require a biopsy or sample of the tumour. This is often an invasive procedure, and there is always the possibility that the relevant cells (i.e. the cells harbouring the relevant mutation) are not taken by the biopsy, or are taken in a low frequency compared to other cells, meaning that particular mutations may be missed.

However, recently it has been appreciated that tumour DNA can be found in the blood of patients, so called circulating tumour DNA (ctDNA). Tests that are capable of taking advantage of this are therefore desired. However, ctDNA is very difficult to work with. It is generally degraded, of low abundance, and mixed with wild-type DNA. Commercial kits, if not designed specifically for ctDNA, are not suitable as they require high input of high quality DNA and may focus on long regions (e.g. 200-250 bp), thus excluding the amplification of shorter (degraded) molecules. Finally, if commercial tests are not designed to favour amplification of mutant sequences, they are not sensitive enough to work with ctDNA.

The tests of the present invention are considered to have numerous advantages over standard commercial tests, for example the Roche™ test. Although the Roche™ test picks up the vast majority of mutations in the KRAS codons 12 and 13, the tests of the present invention are considered to be capable of picking up additional, low frequency mutations that although they are rare, should not be ignored. It is also possible that the low frequency alleles may be more prevalent than currently thought, due to disproportionate focus on the more frequent alleles, making the tests of the invention even more relevant. In addition, the tests of the invention work well, and actually work better on ctDNA, whilst the Roche™ test does not. The tests of the present invention identify all mutations within the region of interest whilst the Roche™ test only works on selected mutations. Furthermore, the lower limit of detection of the presently claimed tests is <0.1% tumour DNA, whilst the Roche™ test is far less sensitive and can only detect approximately as low as 5% tumour DNA.

Various PCR techniques have been specifically developed to enable the preferential amplification, and therefore detection, of low frequency mutant alleles. These techniques include COLD-PCR in which the mutant and wildtype alleles hybridise to form a heteroduplex, which, in most cases, has a slightly lower melting temperature than the wildtype homoduplex, or the mutant allele exhibits lower melting temperature than the wildtype one.

Li et al 2009 Biochem Soc Trans 37: 427-432, discusses the COLD-PCR technique and exemplifies the technique using exon 8 mutations of p53, with a Tc of 86.5, which enriched the mutant from 5% to 65%.

Milbury et al 2010 NAR 39: No. 1, discusses Ice-COLD-PCR, exemplified using the p53 exon 8 mutations from cancer cell-lines HCC2218 and HCC1008. FAST-COLD PCR was shown to only enrich mutations that lower the Tm, whilst Ice-COLD-PCR is useful for those mutations that do not affect the Tm, or that raise it, and so is considered useful when assessing unknown mutations.

Li et al 2009 Clin Chem 55: 748-756 teaches that COLD-PCR enhances the mutation detection selectivity of TaqMan based Real Time PCR. This was exemplified by the p53 exon 8 mutation in codon 273 (G>A).

Mancini et al (2010) J Mol Diagn 12(5) 705-711 discusses the use of COLD-PCR, exemplified by the amplification of the KRAS codons 12 and 13.

Wu et al (2014) Asian Pac J Cancer Prev 15(24) 10647-52—used COLD-PCR to amplify across the KRAS codons 12 and 13.

We provide herein sets of primers and particular reaction conditions for the detection of mutations in the KRAS gene, particularly mutations in codons 12 and 13; mutations in the p53 gene, particularly mutations in exons 5, 6, 7 and 8; and mutations in EGFR exon 19 and 21. The primers are considered to be particularly useful when used with COLD-PCR under the conditions described herein. However, the primers may be used in any reaction with any conditions. The agents and conditions all the identification of mutations using, for example, high resolution melt curve analysis.

Furthermore, the agents and conditions of the present invention have been found to be particularly advantageous when used in combination with circulating tumour DNA, such as tumour DNA obtained from a blood sample for example wherein the circulating tumour DNA is in the plasma and results in increased sensitivity and enhanced detection rates over the tests of the prior art, such as the Roche™ Cobas™ tests. The ease and negligible cost of obtaining a blood sample relative to obtaining a tumour biopsy (which may be from £300 to £3000 depending on if the biopsy is a CT or surgical biopsy), combined with the enhanced sensitivity, results in the agents and conditions of the present invention representing large advances over the prior art tests.

### SUMMARY OF THE INVENTION

The inventors have identified and optimised a novel combination of nucleic acids and reaction conditions that allow the detection of multiple mutations in the KRAS, p53 or EGFR genes from a simple blood sample taken from a subject. The convenience of such an assay is considered to both speed up diagnosis and choice of therapy, whilst alleviating discomfort for the subject. Moreover, the presently claimed methods and reagents are capable to detecting certain mutations that cannot possibly be detected by current standard approved tests. Approximately 15-25% of patients with lung adenocarcinoma have tumor associated KRAS mutations. KRAS mutations are uncommon in lung squamous cell carcinoma (Brose et al. 2002). In the majority of cases, these mutations are missense mutations which introduce an amino acid substitution at position 12, 13, or 61. The result of these mutations is constitutive activation of KRAS signaling pathways.

In the vast majority of cases, KRAS mutations are found in tumors wild type for EGFR or ALK; in other words, they are non-overlapping with other oncogenic mutations found in

NSCLC. Therefore, KRAS mutation defines a distinct molecular subset of the disease. KRAS mutations are found in tumors from both former/current smokers and never smokers. They are rarer in never smokers and are less common in East Asian vs. US/European patients (Riely et al. 2008; Sun et al. 2010).

The role of KRAS as either a prognostic or predictive factor in NSCLC is unknown at this time. Very few prospective randomized trials have been completed using KRAS as a biomarker to stratify therapeutic options in the metastatic setting. Unlike in colon cancer, KRAS mutations have not yet been shown in NSCLC to be negative predictors of benefit to anti-EGFR antibodies. However, KRAS mutations are negative predictors of radiographic response to the EGFR tyrosine kinase inhibitors, erlotinib and gefitinib [for review, see (Riely and Ladanyi 2008; Riely, Marks, and Pao 2009)]. Currently, there are no direct anti-KRAS therapies available.

Approximately 15-25% of patients with lung adenocarcinoma have tumor associated KRAS mutations. KRAS mutations are uncommon in lung squamous cell carcinoma (Brose et al. 2002). In the majority of cases, these mutations are missense mutations which introduce an amino acid substitution at position 12, 13, or 61. The result of these mutations is constitutive activation of KRAS signaling pathways.

In the vast majority of cases, KRAS mutations are found in tumors wild type for EGFR or ALK; in other words, they are non-overlapping with other oncogenic mutations found in NSCLC. Therefore, KRAS mutation defines a distinct molecular subset of the disease. KRAS mutations are found in tumors from both former/current smokers and never smokers. They are rarer in never smokers and are less common in East Asian vs. US/European patients (Riely et al. 2008; Sun et al. 2010).

The role of KRAS as either a prognostic or predictive factor in NSCLC is unknown at this time. Very few prospective randomized trials have been completed using KRAS as a biomarker to stratify therapeutic options in the metastatic setting. Unlike in colon cancer, KRAS mutations have not yet been shown in NSCLC to be negative predictors of benefit to anti-EGFR antibodies. However, KRAS mutations are negative predictors of radiographic response to the EGFR tyrosine kinase inhibitors, erlotinib and gefitinib [for review, see (Riely and Ladanyi 2008; Riely, Marks, and Pao 2009)]. Currently, there are no direct anti-KRAS therapies available.

Approximately 15-25% of patients with lung adenocarcinoma have tumor associated KRAS mutations. KRAS mutations are uncommon in lung squamous cell carcinoma (Brose et al. 2002). In the majority of cases, these mutations are missense mutations which introduce an amino acid substitution at position 12, 13, or 61. The result of these mutations is constitutive activation of KRAS signaling pathways.

Therefore the present invention ensures a more accurate diagnosis of the mutation status of the KRAS, p53 or EGFR genes in a subject, allowing more defined tailoring of therapy.

### DETAILED DESCRIPTION OF THE INVENTION

In a first aspect the invention provides a chemically synthesized nucleic acid of less than 50 nucleotides in length comprising any one of the following sequences [SEQ ID NO: 1 to SEQ ID NO: 21]:

| Locus | Forward | Reverse |
|---|---|---|
| KRAS | AAAACAAGATTTACCT | AGGCCTGCTGAAAATGACTG |
| | CTATTGTTGGA | [SEQ ID NO: 2] |
| | [SEQ ID NO: 1] | |
| EGFR exon 19 | GTTAAAATTCCCGTCG | GACCCCCACACAGCAAA |
| (set 2) | CTATCA | [SEQ ID NO: 4] |
| | [SEQ ID NO: 3] | |
| EGFR exon 19 | AAGTTAAAATTCCCGT | GACCCCCACACAGCAAA |
| (set 3) | CGCTATC | [SEQ ID NO: 4] |
| | [SEQ ID NO: 5] | |
| EGFR exon 21 | GCAGCATGTCAAGATC | TGCCTCCTTCTGCATGGTAT |
| | ACAGA | |
| | [SEQ ID NO: 6] | [SEQ ID NO: 7] |
| P53 exon 5-1 | AACCAGCCCTGTCGTC | CAAGCAGTCACAGCACATGA |
| | TCT | |
| | [SEQ ID NO: 8] | [SEQ ID NO: 9] |
| P53 exon 5-2 | CTGAGCAGCGCTCATG | GTGCAGCTGTGGGTTGATTC |
| | GT | |
| | [SEQ ID NO: 10] | [SEQ ID NO: 11] |
| P53 exon 5-3 | GGGGGTGTGGAATCAA | ACTTGTGCCCTGACTTTCAA |
| | C | |
| | [SEQ ID NO: 12] | [SEQ ID NO: 13] |
| P53 exon 6 | CAGTTGCAAACCAGAC | GGCCTCTGATTCCTCACTGAT |
| | CTCA | |
| | [SEQ ID NO: 14] | [SEQ ID NO: 15] |
| P53 exon 7 | GGCTCCTGACCTGGAG | CTTGGGCCTGTGTTATCTCC |
| | TCTT | |
| | [SEQ ID NO: 16] | [SEQ ID NO: 17] |
| P53 exon 8-1 | TCTTGCGGAGATTCTC | GCCTCTTGCTTCTCTTTTCCT |
| | TTCC | |
| | [SEQ ID NO: 18] | [SEQ ID NO: 19] |
| P53 exon 8-2 | GCTTCTTGTCCTGCTT | CTACTGGGACGGAACAGCTT |
| | GCTT | |
| | [SEQ ID NO: 20] | [SEQ ID NO: 21] |

In one embodiment the invention provides a chemically synthesized nucleic acid consisting of any one of the above sequences (SEQ ID NO: 1 to SEQ ID NO: 21).

The nucleic acid molecule may be DNA or RNA, and is preferably DNA. It may comprise deoxyribonucleotides, ribonucleotides, modified nucleotides or bases, and/or their analogues, or any substrate that can be incorporated into a polymer by DNA or RNA polymerase, or by a synthetic reaction. A polynucleotide may comprise modified nucleotides, such as methylated nucleotides and their analogues. If present, modification to the nucleotide structure may be imparted before or after assembly of the polymer. The sequence of nucleotides may be interrupted by non-nucleotide components. The nucleic acid may be a peptide nucleic acid.

In a preferred embodiment the nucleic acid is DNA. The nucleic acid may be considered to be a primer and is capable of extension during the polymerase chain reaction.

The nucleic acid may also be modified with detectable agents, such as fluorophores, or reactive pairs such as a fluorophore/quencher pair.

The nucleic acids of the invention may have the sequences of SEQ ID NO: 1-21, or the nucleic acids may be similar to those sequences, for example the nucleic acid may have at least 60% similarity to any of SEQ ID NO: 1-21, for example at least 65% similarity, for example at least 70% similarity, for example at least 75% similarity, for example at least 80% similarity, for example at least 85% similarity, for example at least 90% similarity, for example at least 92% similarity, for example at least 94% similarity, for example at least 95% similarity, for example at least 96% similarity, for example at least 97% similarity, for example at least 98% similarity, for example at least 99% similarity, for example 100% similarity.

In one embodiment, the nucleic acids of the invention have the same or similar length as the sequences of SEQ ID NO: 1-21, for example may be 1, 2, 3, 4, 5, 6, 7, 8, 9 or 10 or more nucleic acids longer or shorter than the sequences of SEQ ID NO: 1-21, and are similar in sequence to those sequences, for example the nucleic acid may have at least 60% similarity to any of SEQ ID NO: 1-21, for example at least 65% similarity, for example at least 70% similarity, for example at least 75% similarity, for example at least 80% similarity, for example at least 85% similarity, for example at least 90% similarity, for example at least 92% similarity, for example at least 94% similarity, for example at least 95% similarity, for example at least 96% similarity, for example at least 97% similarity, for example at least 98% similarity, for example at least 99% similarity, for example 100% similarity.

In one embodiment, the sequence of the nucleic acid is 100% identical to that of any of SEQ ID NO: 1-21, but the sequence may be longer or shorter. Or the sequence may have a lower level of similarity and be of the same or different length as the sequences of SEQ ID NO: 1-21.

In one embodiment, the nucleic acids of the invention consist of any one of the sequences of SEQ ID NO: 1-21.

In any event, the sequences of the nucleic acids of the invention must be capable of hybridising to the corresponding complementary sequences, which will be present in the target DNA sample. In one embodiment the nucleic acid of the invention hybridises specifically to the corresponding target sequence. The skilled person will appreciate that any given sequence can tolerate a number of mismatches between the target sequence and the nucleic acid, for example between a target sequence and a primer. The skilled person will know that as the number of mismatches increases so does the chances of the primer either not binding at all to the correct position in the target sequence, and/or also binding elsewhere in the target sequences, leading to non-specific priming and undesired extension products. The skilled person would be aware of adjusting parameters such as annealing temperature and magnesium concentration within the reaction mix in order to mitigate such events. Therefore in one embodiment the nucleic acid can have any level of sequence similarity to the target sequence, provided it is capable of hybridising to the target, and the skilled person is well equipped to take such action required to ensure this.

In one embodiment, the sequences of the nucleic acids of the invention are substantially complementary to the target, meaning that the sequences are complementary except for minor regions of mismatch.

Typically, the total number of mismatched nucleotides over a hybridizing region is not more than 3 nucleotides for sequences about 15 nucleotides in length. Conditions under which only exactly complementary nucleic acid strands will hybridize are referred to as “stringent” or “sequence-specific” hybridization conditions. Stable duplexes of substantially complementary nucleic acids can be achieved under less stringent hybridization conditions. Those skilled in the art of nucleic acid technology can determine duplex stability empirically considering a number of variables including, for example, the length and base pair concentration of the oligonucleotides, ionic strength, and incidence of mismatched base pairs. For example, computer software for calculating duplex stability is commercially available from National Biosciences, Inc. (Plymouth, Minn.); e.g., OLIGO version 5, or from DNA Software (Ann Arbor, Mich.), e.g., Visual OMP 6.

Stringent, sequence-specific hybridization conditions, under which an oligonucleotide will hybridize only to the target sequence, are well known in the art. Stringent conditions are sequence-dependent and will be different in different circumstances. Generally, stringent conditions are selected to be about 5° C. lower to 5° C. higher than the thermal melting point (Tm) for the specific sequence at a defined ionic strength and pH.

The Tm is the temperature (under defined ionic strength and pH) at which 50% of the duplex strands have dissociated. Relaxing the stringency of the hybridizing conditions will allow sequence mismatches to be tolerated; the degree of mismatch tolerated can be controlled by suitable adjustment of the hybridization conditions.

In a preferred embodiment, the nucleic acids of the present invention are considered to be primers.

The term “primer” refers to an oligonucleotide that acts as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably about 15 to about 35 nucleotides in length. The isolated nucleic acid of the invention can have a length therefore of at least 12 nucleotides, for example between 12 nucleotides and 40 nucleotides, for example between 14 nucleotides and 38 nucleotides, for example between 16 nucleotides and 36 nucleotides, for example between 18 nucleotides and 34 nucleotides, for example between 19 nucleotides and 32 nucleotides, for example between 20 nucleotides and 30 nucleotides, for example between 21 nucleotides and 29 nucleotides, for example between 22 nucleotides and 28 nucleotides, for example between 23 nucleotides and 27 nucleotides, for example between 24 and 26 nucleotides, for example 25 nucleotides in length.

A primer oligonucleotide can either consist entirely of the hybridizing region or can contain additional features which allow for the detection, immobilization, or manipulation of the amplified product, but which do not alter the ability of the primer to serve as a starting reagent for DNA synthesis. For example, a nucleic acid sequence tail can be included at the 5′ end of the primer that hybridizes to a capture oligonucleotide.

The melting temperature (Tₘ) of an oligonucleotide is the temperature in ° C. at which 50% of the molecules in a population of a single-stranded oligonucleotide are hybridised to their complementary sequence and 50% of the molecules in the population are not-hybridised to said complementary sequence. The Tₘ may be determined empirically, for example Tₘ may be measured using melting or annealing curve analysis, e.g. using a Bio-Rad CFX instrument on a 96-well white plate. The Tₘ of an oligonucleotide probe is the temperature point of greatest rate of change of fluorescence with temperature between the hybridised and non-hybridised states on the probe. Melting peaks may be generated from melting curve data by (−dF/dT). The melting temperature is fundamentally determined by the temperature of a solution containing the oligonucleotides being slowly raised, while continuously observing a fluorescence signal, in order to construct a graph of the negative derivative of fluorescence signal intensity with respect to temperature (−dF/dT) against temperature. The melting temperature (Tₘ) of the hybrid appears as a peak, and provides information about the sequence of the polynucleotide target. The Tₘs generated through melting analysis of the oligonucleotide of the invention may be used to distinguish polymorphic targets.

Where reference is made to a Tₘ for hybridisation involving part of an oligonucleotide, the relevant Tₘ is considered to be the Tₘ that can be calculated from a nearest neighbour analysis of the sequence involved.

The primers of the present invention typically have a melting temperature of between 56° C. and 63° C., for example between 57 and 62° C., for example between 58 and 61° C., for example between 59 and 60° C.

In addition to their use as primers, the nucleic acids of the present invention may also be used in a sequencing reaction, or they may be used as probes.

The nucleic acids of the present invention are intended to be used to amplify across particular regions of the KRAS gene, the p53 gene or the EGFR gene, wherein the regions are known to be such that a mutation in that region is a sign of cancer, or predictor of the development of cancer, or predictor of the likelihood of response to a particular treatment, or wherein a mutation in that region means that a particular treatment should be employed.

Therefore, in a further aspect, the invention provides:

A method of detecting the presence of a mutation in the KRAS gene, and/or the p53 gene, and/or the EGFR gene in a sample obtained from a subject, comprising amplification of the region of interest using any two of the nucleic acids as described in the first aspect, optionally wherein:

[SEQ ID NO: 1]
AAAACAAGATTTACCTCTATTGTTGGA
and
[SEQ ID NO: 2
AGGCCTGCTGAAAATGACTG
Are used to amplify the KRAS locus;
[SEQ ID NO: 3]
GTTAAAATTCCCGTCGCTATCA
and
[SEQ ID NO: 4]
GACCCCCACACAGCAAA
are used to amplify the EGFR exon 19 locus;
[SEQ ID NO: 5]
AAGTTAAAATTCCCGTCGCTATC
and
[SEQ ID NO: 4]
GACCCCCACACAGCAAA
are used to amplify the EGFR exon 19 locus;
[SEQ ID NO: 6]
GCAGCATGTCAAGATCACAGA
and
[SEQ ID NO: 7]
TGCCTCCTTCTGCATGGTAT
are used to amplify the EGFR exon 21 locus;
[SEQ ID NO: 8]
AACCAGCCCTGTCGTCTCT
and
[SEQ ID NO: 9]
CAAGCAGTCACAGCACATGA
are used to amplify the p53 exon 5-1 locus;
[SEQ ID NO: 10]
CTGAGCAGCGCTCATGGT
and
[SEQ ID NO: 11]
GTGCAGCTGTGGGTTGATTC
are used to amplify the p53 exon 5-2 locus;
[SEQ ID NO: 12]
GGGGGTGTGGAATCAAC
and
[SEQ ID NO: 13]
ACTTGTGCCCTGACTTTCAA
are used to amplify the p53 exon 5-3 locus;
[SEQ ID NO: 14]
CAGTTGCAAACCAGACCTCA
and
[SEQ ID NO: 15]
GGCCTCTGATTCCTCACTGAT
are used to amplify the p53 exon 6 locus;
[SEQ ID NO: 16]
GGCTCCTGACCTGGAGTCTT
and
[SEQ ID NO: 17]
CTTGGGCCTGTGTTATCTCC
are used to amplify the p53 exon 7 locus;
[SEQ ID NO: 18]
TCTTGCGGAGATTCTCTTCC
and
[SEQ ID NO: 19]
GCCTCTTGCTTCTCTTTTCCT
are used to amplify the p53 exon 8-1 locus; and
[SEQ ID NO: 20]
GCTTCTTGTCCTGCTTGCTT
and
[SEQ ID NO: 21]
CTACTGGGACGGAACAGCTT
are used to amplify the p53 exon 8-2 locus.

Preferences for the nucleic acids in relation to the earlier aspect also apply to this embodiment, for example wherein for example SEQ ID NO: 1 is discussed, we include the meaning of all the features of aspect 1, for example we include the meaning of sequences that are similar to that of SEQ ID NO:1.

The sample may be any sample obtained from a subject. The sample may be processed, for example fixed in formaldehyde and/or embedded in paraffin. Preferably the sample is a fresh sample. The sample may be a biopsy taken from a tumour, for example from a solid tumour. Alternatively, for example wherein the cancer is a blood cancer, the sample may be a sample of affected blood cells. However, it is considered particularly advantageous, both in terms of the data obtained and the comfort of the subject, if the sample is a blood, plasma or serum sample, and the target DNA is circulating tumour DNA.

The blood, plasma or serum sample may be processed prior to amplification of the target, or may be used unprocessed. The plasma or serum is ideally obtained from fresh unfrozen blood, ideally within 30 minutes upon blood acquisition. However, older blood can also be used. In one embodiment, the circulating tumour DNA can be extracted from plasma, for example from 9 ml of peripheral blood from a subject. In some embodiments, the sample, for example the serum or plasma sample may have been stored prior to extraction of the circulating tumour DNA or prior to direct analysis, for example may have been stored at −80° C. The blood sample can be stored in Cell-free BCT Streck tubes at +4 C. The DNA may be extracted by any number of means known to the skilled person, including the use of commercially available kits, such as the Qiagen QIAmp DNA Blood Mini Kit. Concentration and quality of the DNA can be assessed by techniques known to the skilled person, for example via the use of a spectrophotometer, such as a NanoDrop™ Lite from Thermo Scientific, or by custom designed quantitative real-time PCR, or by fluorometer.

The subject is a human individual including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

The subject may already have been diagnosed with having cancer or may have not yet received a diagnosis, in which case the method of the invention is a diagnostic method. The subject may have been diagnosed with a form of cancer, but not yet begun any cancer therapy, in which case the method may be used to identify the most appropriate treatment based on the presence or absence of the particular mutations. The subject may have begun treatment, but may be such that the treatment is not particularly effective or is not effective at all, in which case it is desired that a more appropriate treatment is found.

The subject may already be known to have any type of cancer, and the method may be for the assessment of the presence of a mutation in any type of cancer. For example the cancer may be benign or metastatic, it may be a primary cancer or a secondary cancer. The cancer may be a solid tumour or a blood borne tumour. The cancer may relate to diseases of skin tissues, organs, blood, and vessels, such as cancers of the bladder, bone, blood, brain, breast, cervix, chest, colon, endometrium, oesophagus, eye, gastrointestinal tract, head, kidney, liver, lymph nodes, lung, mouth, neck, ovaries, pancreas, prostate, rectum, stomach, testis, throat, and uterus.

It will be appreciated that the subject may be one that has had other genetic or other diagnostic tests performed, for example clinical examination. These additional tests may be performed prior to the determination of mutations in the KRAS, p53 and/or EGFR gene, as disclosed herein, may be performed simultaneously, or may be performed following the methods of the invention.

In certain embodiments the cancer is a blood borne cancer. The blood borne cancer may be metastatic. Examples of blood borne cancers include Hodgkin's and Non-Hodgkin's Lymphoma, Burkitt's lymphoma, myeloma or lymphomas, Leukaemia and plasma cell neoplasm. Chromosome 17p genetic diseases also include blood borne cancers such as promyelocytic leukaemia which has a translocation at 17p.

In other embodiments, the cancer is a solid tumour. The solid tumour may be metastatic.

An example of a solid tumour includes inflammatory breast cancer, neuroblastoma, uterine corpus, mature b-cell neoplasm, endocervical carcinoma, endocervicitis, sinus cancer, sclerosing adenosis of breast, maxillary sinus cancer, bronchiolo-alveolar adenocarcinoma, vulva basal cell carcinoma, diffuse large b-cell lymphoma of the central nervous system, chromosome 17p deletion diseases, cerebral primitive neuroectodermal tumor, medullomyoblastoma, large-cell, immunoblastic, primitive neuroectodermal tumor, osteosarcoma, somatic childhood medulloblastoma, plasma cell neoplasm, hepatic angiomyolipoma, Retinoblastoma, melanoma, small cell lung cancer and lung cancer myeloma.

The mutations identifiable by the nucleic acids of the present invention are not confined to any particular cancer or subtype of cancer. For example any cancer may have any of the mutations identifiable by the nucleic acids of the present invention. Thus in one embodiment, the cancer is any type of cancer. Some types of cancer do however show an increased disposition to acquiring certain mutations. It is considered that the mutations identifiable by the nucleic acids of the present invention are particularly prevalent in lung cancer, for example non-small cell lung cancer, and in colorectal cancer or ovarian cancer. Accordingly, in preferred embodiments, the cancer is lung cancer, for example non-small cell lung cancer, or the cancer is colorectal cancer or ovarian cancer.

In one embodiment, the type of cancer is unknown, and the presence of a mutation identifiable by the nucleic acids of the present invention indicates only that the subject has cancer, or is likely to have cancer, but not what the type of cancer is, or where it is.

By detecting the presence of a mutation we include the meaning of detecting a single mutation in that region of the particular gene. We also include the meaning of detecting more than one mutation in that region, for example detecting 2 or more mutations, for example between 2 and 15 or more mutations. The mutation that is detected may be a specific mutation, for example wherein it is known that a particular region may harbour several different mutations, the detecting may be aimed at detecting a single specific mutation. The detecting encompasses the detection of the mere presence or absence of one or more mutations, and also encompasses situations wherein the detecting is capable of identifying the particular mutation, for example by subsequent sequencing of the relevant amplification product.

There are several ways of detecting the presence of a particular mutation, most of which require the prior amplification of the region, particularly when the frequency of the mutation is low, for example wherein a biopsy sample only contains a small percentage of mutated or potentially mutated cells, or wherein the sample is a blood, plasma or serum sample, wherein the target DNA is only a small fraction of the entire DNA. The most commonly used method is the polymerase chain reaction, as will be well known to the skilled person. Therefore, in one embodiment, the method of detecting the presence of a particular mutation comprises an amplification step.

Advances in PCR techniques have led to the development of reaction cycles and conditions that favour the amplification of low frequency mutant alleles.

It is considered that the nucleic acids of the present invention have a particularly advantageous effect when used with such techniques. For example, full COLD-PCR is a technique in which the sample comprising both the low frequency mutant alleles and the wild type high frequency allele is denatured and cooled, allowing the mutant and wildtype single strands to hybridise, leading to the formation of a number of heteroduplexes. The heteroduplexes are in most cases likely to have a slightly different melting temperature, due to the at least one base pair mismatch. The heteroduplex may have no change in melting temperature, or may have an increase in melting temperature. Generally, the heteroduplex will have a slightly lower melting temperature than the wild type homoduplex. This slight difference advantageously means that a PCR cycle employing a particular denaturing temperature (Tc) that is sufficient to denature the heteroduplex but not the homoduplex, allows the preferential amplification of the low frequency mutant allele. At the end of multiple rounds of PCR the frequency of the mutant allele may be increased up to 100 fold from the initial concentration. Therefore in a preferred embodiment the region of interest is amplified using a full COLD-PCR technique. Techniques similar to COLD-PCR including fast-COLD-FOR and Ice-COLD-FOR are also encompassed in the invention.

The skilled person will be well aware as to how to calculate the actual reaction conditions for a given pair of nucleic acid sequences, for example for a given pair of primers. When the PCR is full COLD-PCR, or fast COLD-PCR, or Ice-COLD-FOR, calculation of the Tc is critical. The skilled person would be able to determine this as matter of routine, for example by the use of melt curve analysis. The high-resolution melt analysis can be used to determine the Tc. In such a case, one runs a conventional PCR reaction with the primers of interest and then looks at the maximum value of the melting curve corresponding to the specific amplified fragment. This value is an estimate for Tm, and the Tc is then calculated as Tm −0.5-1.5° C. A range of Tc should then be tested in COLD-PCR reaction to find the Tc resulting in the highest enrichment of mutant alleles.

It will be appreciated that the Tc depends upon the sequence and length of the two primers. The Tc is typically between about 70.0° C. and 90.0° C., for example between 72.0° C. and 88.0° C., for example between 74.0° C. and 86.0° C., for example between 76.0° C. and 84.0° C., for example between 78.0° C. and 82.0° C., for example 80.0° C. and 82.0° C. In a preferred embodiment the Tc is 79.5° C.

The skilled person will be aware that the actual concentrations of the various reagents in the PCR mixture can vary, for example magnesium concentration and primer concentration. Most vendors provide master-mixes containing optimal concentrations of reagents, and recommendations for concentration of primers and input DNA. For example, Type-it HRM PCR kit from Qiagen™ is supplied with a master-mix containing a proprietary HotStartTaq Plus DNA Polymerase (2.5 units per reaction), Q-solution, dNTPs (200 μM of each final), and MgCl₂ (1.5 mM final). The master mix also contains reaction buffer comprising optimal mixture of Tris-CI, KCl, (NH₄)₂SO₄. The recommended concentration of primers to be used with this master mix is 700 nM; however, it can be adjusted experimentally. Usually, 700 nM is optimal.

Exemplary reaction cycles for full COLD-PCR are given in the examples.

The precise temperature chosen for the Tc is considered to be particularly important in providing the optimal results (see the Examples wherein even half a degree C. difference can affect the results). In one embodiment therefore:
where the target is KRAS and the nucleic acids are AAAACAAGATTTACCTCTATTGTTGGA [SEQ ID NO:1] and AGGCCTGCTGAAAATGACTG [SEQ ID NO: 2], the Tc of the COLD-PCR is 79° C., optionally wherein the denaturing time is 3 seconds;
where the target is EGFR and the nucleic acids are GTTAAAATTCCCGTCGCTATCA [SEQ ID NO: 3] and GACCCCCACACAGCAAA [SEQ ID NO: 4], the Tc of the COLD-PCR is 78.5° C.;
where the target is EGFR and the nucleic acids are AAGTTAAAATTCCCGTCGCTATC [SEQ ID NO: 5] and GACCCCCACACAGCAAA [SEQ ID NO: 4], the Tc of the COLD-PCR is 78.5° C.;
where the target is EGFR and the nucleic acids are GCAGCATGTCAAGATCACAGA [SEQ ID NO: 6] and TGCCTCCTTCTGCATGGTAT [SEQ ID NO: 7] the Tc of the COLD-PCR is 86.5° C.;
where the target is p53 and the nucleic acids are AACCAGCCCTGTCGTCTCT [SEQ ID NO: 8] and CAAGCAGTCACAGCACATGA [SEQ ID NO: 9] the Tc of the COLD-PCR is 86.7° C., optionally wherein the denaturing time is 10 seconds;
where the target is p53 and the nucleic acids are CTGAGCAGCGCTCATGGT [SEQ ID NO: 10] and GTGCAGCTGTGGGTTGATTC [SEQ ID NO: 11] the Tc of the COLD-PCR is 89° C., optionally wherein the denaturing time is 10 seconds;
where the target is p53 and the nucleic acids are GGGGGTGTGGAATCAAC [SEQ ID NO: 12] and ACTTGTGCCCTGACTTTCAA [SEQ ID NO: 13] the Tc of the COLD-PCR is 83° C., optionally wherein the denaturing time is 10 seconds;
where the target is p53 and the nucleic acids are CAGTTGCAAACCAGACCTCA [SEQ ID NO: 14] and GGCCTCTGATTCCTCACTGAT [SEQ ID NO: 15] the Tc of the COLD-PCR is 87.5° C., optionally wherein the denaturing time is 3 seconds;
where the target is p53 and the nucleic acids are GGCTCCTGACCTGGAGTCTT [SEQ ID NO: 16] and CTTGGGCCTGTGTTATCTCC [SEQ ID NO: 17] the Tc of the COLD-PCR is 83.5° C., optionally wherein the denaturing time is 10 seconds;
where the target is p53 and the nucleic acids are TCTTGCGGAGATTCTCTTCC [SEQ ID NO: 18] and GCCTCTTGCTTCTCTTTTCCT [SEQ ID NO: 19] the Tc of the COLD-PCR is 81.8° C., optionally wherein the denaturing time is 20 seconds;
where the target is p53 and the nucleic acids are GCTTCTTGTCCTGCTTGCTT [SEQ ID NO: 20] and CTACTGGGACGGAACAGCTT [SEQ ID NO: 21] the Tc of the COLD-PCR is 85.3° C., optionally wherein the denaturing time is 10 seconds;

It will however be appreciated that the optimal Tc, and other reaction conditions may be slightly different if different reaction equipment and different reaction components are used. Thus it will be appreciated that where a precise Tc value is given, this is intended to cover a close range of possible Tc's that may be optimal under different circumstances. For example reference to a Tc value of 79° C., for example for one machine/step-up, may correspond to, and is intended to cover, a slightly different temperature with a different machine or set-up, for example from 78° C. to 80° C.; similarly 78.5° C. may correspond to, and is intended to cover from 77.5° C. to 79.5° C.; 86.5° C. may correspond to, and is intended to cover from 85.5° C. to 87.5° C.; 86.7° C. may correspond to, and is intended to cover from 85.7° C. to 87.7° C.; 89° C. may correspond to, and is intended to cover from 88° C. to 90° C.; 83° C. may correspond to, and is intended to cover from 82° C. to 84° C.; 87.5° C. may correspond to, and is intended to cover from 86.5° C. to 88.5° C.; 83.5° C. may correspond to, and is intended to cover from 82.5° C. to 84.5° C.; 81.8° C. may correspond to, and is intended to cover from 80.8° C. to 82.8° C.; and 85.3° C. may correspond to, and is intended to cover from 84.3° C. to 86.3° C.; depending on the reaction set up.

In a further exemplary embodiment, which may be used in conjunction with any reaction cycle, including those given in the examples, the amplification, for example COLD-PCR is carried out using the Type-it HRM PCR kit from Qiagen. The PCR reaction mix comprises a final concentration of 1× Type-it HTM mix and 700 nm of each primer. The range of DNA concentrations used with these conditions is typically 0.1 ng-10 ng.

In particular embodiments, the reaction conditions are as follows:

a)

| Stage | Temp | Time | Cycles |
|---|---|---|---|
| Activation | 95° C. | 5 min | 1 |
| Pre-PCR | 95° C. | 10 sec | 20 |
| | 55° C. | 30 sec | |
| | 72° C. | 10 sec | |
| COLD | 79° C. | 3 sec | 40/45 |
| | 55° C. | 30 sec | |
| | 72° C. | 10 sec | |
| HRM | 68-90 | | 1 |

where the target is KRAS and the reaction is fast COLD-PCR;

b)

| Stage | Temp | Time | Cycles |
|---|---|---|---|
| Activation | 95° C. | 5 min | 1 |
| Pre-PCR | 95° C. | 10 sec | 20 |
| | 60° C. | 1 min | |
| COLD | 95° C. | 10 sec | 40/45 |
| | 70° C. | 5 min | |
| | 79.5° C. | 3 sec | |
| | 55° C. | 30 sec | |
| | 72° C. | 10 sec | |
| HRM | 68-90 | | 1 |

where the target is KRAS and the reaction in full COLD-PCR;

c)

| Stage | Temp | Time | Cycles |
|---|---|---|---|
| Activation | 95° C. | 5 min | 1 |
| Pre-PCR | 95° C. | 10 sec | 20 |
| | 55° C. | 30 sec | |
| | 72° C. | 10 sec | |
| COLD | 78.5° C. | 3 sec | 40/45 |
| | 55° C. | 30 sec | |
| | 72° C. | 10 sec | |
| HRM | 68-90 | | 1 |

where the target is EGFR exon 19 and the reaction is fast COLD-PCR;

d)

| Stage | Temp | Time | Cycles |
|---|---|---|---|
| Activation | 95° C. | 5 min | 1 |
| Pre-PCR | 95° C. | 10 sec | 20 |
| | 60° C. | 1 min | |
| COLD | 95° C. | 10 sec | 40/45 |
| | 70° C. | 5 min | |
| | 86° C. | 3 sec | |
| | 55° C. | 30 sec | |
| | 72° C. | 10 sec | |
| HRM | 68-90 | | 1 |

where the target is EGFR exon 21 and the reaction is full COLD-PCR;

e)

| Stage | Temp | Time | Cycles |
|---|---|---|---|
| Activation | 95° C. | 5 min | 1 |
| Pre-PCR | 95° C. | 10 sec | 20 |
| | 55° C. | 30 sec | |
| | 72° C. | 10 sec | |
| COLD | Tc° C. | | 40/45 |
| | SEQ ID NO: 8 and 9 - 86.7° C. | 10 sec | |
| | SEQ ID NO: 10 and 11 - 89° C. | 10 sec | |
| | SEQ ID NO: 12 and 13 - 83° C. | 10 sec | |
| | SEQ ID NO: 14 and 15 - 87.5° C. | 3 sec | |
| | SEQ ID NO: 16 and 17 - 83.5° C. | 10 sec | |
| | SEQ ID NO: 18 and 19 - 81.8° C. | 20 sec | |
| | SEQ ID NO: 20 and 21 - 85.3° C. | 10 sec | |
| | 55° C. | 30 sec | |
| | 72° C. | 10 sec | |
| HRM | 68-90 | | 1 |

where the target is p53 and the reaction is fast COLD-PCR.

The skilled person would be aware that following amplification of the relevant region, there are several options for the actual method used to detect whether the amplified sample comprises mutations or not. For example, the presence of a mutation in the amplified products may be assessed by the use a microarray, for example a microarray which comprises probes to each of the possible mutants. In addition to giving information as to whether a mutation is present or not, the actual mutation will also be revealed.

In another embodiment the presence of a mutation in the amplified product may be determined by sequencing the products. This may be done directly following the amplification reaction (with or without various clean-up steps) i.e. prior to any other screening method to determine the presence of a mutation. In this case, the sequencing will reveal in the first instance whether there is a mutation present or not, and secondly what the mutation is. Alternatively, sequencing can be employed following any of the other techniques that will be apparent to the skilled person to determine the presence of a mutation, for example the amplification product can be screened via a microarray or melt curve analysis, and then only those samples that show the presence of a mutation may be sequenced.

In a further embodiment, the use of high resolution melt curve analysis can be used, for example using a Rotorgene (QIAGEN) in a single tube/reaction. Melt curve analysis looks at the temperature required to denature double stranded nucleic acid. The particular temperature at which this occurs, the melting temperature, is dependent upon the sequence and length of a given double stranded nucleic acid. For example, if when using the same set of primers two products are obtained, one in which an AT pair has been replaced with a GC pair, the melting temperature of the latter is likely to be higher, due to the higher amount of energy required to split a GC bond than an AT bond. Such techniques are very sensitive and can be used to distinguish very minor differences between sequences.

The method employing melt curve analysis is semi-quantitative, such that as the number of mutations in a given region increases, the change in melting temperature of the heteroduplex becomes greater and can be identified in the melt curve analysis. Thus in one embodiment, the presence of a mutation in a given sequence is assessed at one time point, as described herein, and again at a subsequent one or more time points, and the resultant melt curves compared to enable the detection of an increase in the presence of mutations. This is considered to aid in an assessment of the subject's prognosis.

In one embodiment, at the later or subsequent time points, the number of mutations identified has increased, and the subject has a worse prognosis.

In another embodiment, at the later or subsequent time points, the number of mutations has decreased, or no mutations are detected at all. In one embodiment this indicates that the cancer has gone. In another embodiment, it indicates that the treatment regime that the subject has undergone has been successful. Thus the invention provides a method for monitoring the efficacy of any particular anti-cancer therapy where the subject is known to have, or have had, cancer which comprises any one or more of the mutations identifiable by the nucleic acids of the present invention.

In one embodiment, the melt curve analysis can be performed over 1 cycle of 60° C. to 95° C.

The melting curves can be analysed by computer software such as High Resolution Melt Software v 3.0.1 (Life technologies).

As stated earlier, once the mutation status of a subject is known, either the presence or absence of any mutation in a given gene, or the identification of particular mutations in a given gene, appropriate treatment can be determined. Therefore in one embodiment the method further comprises the determination of suitable therapy or treatment, based on the mutation profile of the subject as determined by the methods of the invention.

For example, tumours with certain mutations are known to respond, or to not respond, to particular therapies. For example, for cancers that are positive for EGFR mutation, beneficial treatments are considered to be Gefitinib, Erlotinib and Afatanib (for lung cancer). For cancers that are positive for EGFR mutation, non-beneficial treatments are considered to be panitumumab and cetuximab (for colorectal cancer).

For example, if the subject is found to have a mutation in the EGFR gene, a suitable treatment may be considered to be Gefitinib, Erlotinib and Afatanib.

KRAS mutation is predictive of a very poor response to panitumumab (Vectibix®) and cetuximab (Erbitux®) therapy in colorectal cancer. Currently, the most reliable way to predict whether a colorectal cancer patient will respond to one of the EGFR-inhibiting drugs is to test for certain “activating” mutations in the gene that encodes KRAS, which occurs in 30%-50% of colorectal cancers. Studies show patients whose tumours express the mutated version of the KRAS gene will not respond to cetuximab or panitumumab.

In some embodiments the method further comprises administration to the subject of a suitable therapeutic.

It will be appreciated that administration of any agent described herein, for example a therapeutic agent, for example and anti-cancer agent, is typically administered as part of a pharmaceutical composition together with a pharmaceutically acceptable excipient, diluent, adjuvant, or carrier. Thus, any mention of a particular compound or drug, and any mention of a therapeutic agent, equally applies to a pharmaceutically acceptable composition comprising that agent (e.g. a formulation).

The agents such as therapeutics may be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. The agents for example the therapeutics can be administered orally, buccally or sublingually in the form of tablets, capsules, ovules, elixirs, solutions or suspensions, which may contain flavouring or colouring agents, for immediate-, delayed- or controlled-release applications. The therapeutics may also be administered via intracavernosal injection. The skilled person would be aware of the best route of administration for each particular therapeutic that is to be administered. The skilled person would also be aware of a suitable dosage or dosage regime for each particular therapeutic.

The methods of the invention may also further comprise the assessment of the mutation status of another gene or genes, in order to obtain a more accurate and useful picture of the treatment requirements of the subject. For example, it is considered that the chronological order of mutations is important in the impact of KRAS mutations in regard to colorectal cancer, with a primary KRAS mutation generally leading to a self-limiting hyperplastic or borderline lesion, but if occurring after a previous APC mutation it often progresses to cancer (Vogelstein B, Kinzler K W (August 2004). “Cancer genes and the pathways they control”. Nat. Med. 10 (8): 789-99. doi:10.1038/nm1087. PMID 15286780).

Therefore, in one embodiment, the methods of the invention further comprise the assessment of the mutation status of another gene or genes, for example the APC, BRAF, ALK, PIK3CA, DDR2, HER2, FGFR1, MAP2K1, MET, NRAS, NTRK1, PTEN, RET, ROS1 genes. Moreover, in an additional embodiment, the method comprises the temporal assessment of the appearance of mutations. For example, the method may comprise the assessment of the appearance of one or more mutations in the KRAS, p53 and/or EGFR loci, for example by performing the methods of the invention on samples taken from the subject at different time points. For example, in one embodiment, the subject may be known to have a mutation in the APC gene, but may not have a mutation in the KRAS gene and the method of the invention, i.e. the method to identify the presence of a mutation in the KRAS gene, in which case samples may be taken from the subject at for example intervals, which may be regular or irregular intervals, and assessed for the appearance of a KRAS mutation where treatment can be tailored accordingly. Similarly, the subject may be known to have no mutations in the KRAS, p53 and/or EGFR locus, but it is desirable to monitor the subject for the appearance of such mutations. Such methods are also encompassed by the present invention.

Such a method can be used to predict the likelihood of a subject developing cancer. For example the identification of the order in which particular mutations appear can be used to predict whether a subject will develop cancer, and can be used to decide treatment accordingly. For example, the method can be used to predict whether a subject will develop cancer, for example colorectal cancer, wherein the method comprises assessing for the appearance of both a mutation in the KRAS locus and APC locus, the method comprising the determination of the presence of one or more mutations in the KRAS and APC locus in samples taken from a subject at different times.

In all methods, the mutation under which the presence of is being determined, may be a point mutation, a deletion mutation or an insertion mutation. The mutation may be a synonymous or may be a nonsynonymous mutation.

Further methods arising from the particular combination of nucleic acids and mutation detection methods, and also claimed, include a method for determining a subject's suitability for treatment with an anti-EGFR therapy, comprising assessing the presence or absence of a mutation in the EGFR and/or KRAS gene, wherein for example the presence of a mutation is identified using the methods of the invention detailed above. If the subject is found to have a mutation in the EGFR gene the subject is deemed suitable for EGFR therapy. If the subject is found to have a mutation in the KRAS gene, the subject is not deemed to be suitable for anti-EGFR therapy.

Further, we provide a method of treating a subject for cancer, wherein the method comprises determination of the presence or absence of a mutation in the KRAS, p53 and/or EGFR gene, in combination with any other gene as required, wherein the determination is carried out according to the methods disclosed herein, and further comprising the administration of a therapeutic agent, wherein the choice of therapeutic agent is determined based on the determined mutation profile.

A further claimed method is a method of diagnosing a subject with cancer, or a pre-cancerous lesion, comprising the determination of the presence or absence of a mutation in the KRAS, p53 and/or EGFR gene wherein the determination is carried out according to the methods disclosed herein, wherein the presence of a mutation indicates that the subject is likely to have, or to develop, cancer.

Also included in the invention is an anticancer agent, for example Gefitinib, Erlotinib or Afatanib for use in treating cancer, wherein the subject has been determined to be suitable for treatment with Gefitinib, Erlotinib or Afatanib according to the methods of the invention.

The use of an anticancer agent, for example drug Gefitinib, Erlotinib or Afatanib for use in the manufacture of a medicament for use in treating cancer, wherein the subject has been determined to be suitable for treatment with Gefitinib, Erlotinib or Afatanib according to the methods of the invention, is also encompassed.

The invention also provides a kit of parts comprising at least any one of the nucleic acids of the invention. The kit of parts preferably comprises at least any two of the nucleic acids, and preferably comprises at least any two nucleic acids selected from:

[SEQ ID NO: 1]
AAAACAAGATTTACCTCTATTGTTGGA
and
[SEQ ID NO: 2
AGGCCTGCTGAAAATGACTG;
[SEQ ID NO: 3]
GTTAAAATTCCCGTCGCTATCA
and
[SEQ ID NO: 4]
GACCCCCACACAGCAAA;
[SEQ ID NO: 5]
AAGTTAAAATTCCCGTCGCTATC
and
[SEQ ID NO: 4]
GACCCCCACACAGCAAA;
[SEQ ID NO: 6]
GCAGCATGTCAAGATCACAGA
and
[SEQ ID NO: 7]
TGCCTCCTTCTGCATGGTAT;
[SEQ ID NO: 8]
AACCAGCCCTGTCGTCTCT
and
[SEQ ID NO: 9]
CAAGCAGTCACAGCACATGA;
[SEQ ID NO: 10]
CTGAGCAGCGCTCATGGT
and
[SEQ ID NO: 11]
GTGCAGCTGTGGGTTGATTC;
[SEQ ID NO: 12]
GGGGGTGTGGAATCAAC
and
[SEQ ID NO: 13]
ACTTGTGCCCTGACTTTCAA;
[SEQ ID NO: 14]
CAGTTGCAAACCAGACCTCA
and
[SEQ ID NO: 15]
GGCCTCTGATTCCTCACTGAT;
[SEQ ID NO: 16]
GGCTCCTGACCTGGAGTCTT
and
[SEQ ID NO: 17]
CTTGGGCCTGTGTTATCTCC;
[SEQ ID NO: 18]
TCTTGCGGAGATTCTCTTCC
and
[SEQ ID NO: 19]
GCCTCTTGCTTCTCTTTTCCT;
[SEQ ID NO: 20]
GCTTCTTGTCCTGCTTGCTT
and
[SEQ ID NO: 21]
CTACTGGGACGGAACAGCTT.

The kit may comprise any two of the nucleic acids of the invention, optionally any 3, or any 4, or any 5, or any 6, or any 7, or any 8, or any 9, or any 10, or any 11, or any 12, or any 13, or any 14, or any 15, or any 16, or any 17, or any 18, or any 19, or any 20, or any 21 nucleic acids according to the invention.

The kit may comprise all nucleic acids of the invention, or the kit may comprise all nucleic acids directed to one particular gene, for example the kit may comprise nucleic acids SEQ ID NO: 1 and SEQ ID NO: 2 and be for the identification of mutations in the KRAS locus. Or the kit may comprise nucleic acids of SEQ ID NO: 8-21- and be for the identification of mutations in the p53 locus. Or the kit may comprise nucleic acids of SEQ ID NO: 3-7 and be for the identification of mutation in the EGFR locus.

The kit may further comprise one or more control samples, for example wherein the control samples comprise the wild type and/or the mutant version of each PCR product generated by each pair of primers.

The kit may comprise components for standard melting curves generated by each mutant PCR product to which the user may compare the melting curve generated by the sample.

The kit may comprise PCR reagents and/or detection reagents, for example, master-mix for PCR

In one embodiment the kit comprises reaction wells, for example a reaction plate, for example a microtitre plate, or individual tubes. The wells, for example reaction plate, for example microtitre plate, may contain an aliquot of the relevant nucleic acids and/or other PCR reagents. The tubes or wells of a plate may also comprise control DNA pre-disposed in the relevant wells.

Preferences and options for a given aspect, feature or parameter of the invention should, unless the context indicates otherwise, be regarded as having been disclosed in combination with any and all preferences and options for all other aspects, features and parameters of the invention.

The listing or discussion of an apparently prior-published document in this specification should not necessarily be taken as an acknowledgement that the document is part of the state of the art or is common general knowledge.

The invention will now be described with the aid of the following non-limiting figures and examples.

### FIGURE LEGENDS

For all difference curve graphs—The curves on the graph (identified in the graph legends as “variant 1”, “variant 2” etc) represent melting curves for individual tested samples containing different concentrations of mutant DNA in a mixture of mutant and wild-type DNA. The x-axis is for temperature; the y-axis is for the difference in melting profile as compared to a negative control (pure wild-type DNA; y=0). The deviation of a melting curve from the control suggests the presence of a mutation. The higher the concentration of mutant DNA, the higher the deviation from control is expected. Depending on the sensitivity of the method (subject of primer sequences and PCR-conditions), lower or higher percentage of mutations can be detected. For example, in the graph shown in FIG. 1A, the lowest concentration of mutant DNA one can distinguish from the control is ˜10%. In other words, the sensitivity for this primer set was found to be as little as 10% mutant DNA. Analogous approaches are applied for other primer sets and conditions below.

FIG. 1. The mutated DNA comprises a mutation G12S in the KRAS gene.

FIG. 1A. The results of initial assessment of analytic sensitivity of Set1 primers (Tc=Tm−1). The sensitivity was found to be As little as 10% mutant DNA. In this graph the lowest concentration of mutant DNA one can distinguish from the control is −10%. In other words, the sensitivity for this primer set was found to be as little as 10% mutant DNA.

FIG. 1B. The results of initial assessment of analytic sensitivity of Set2 primers (Tc=Tm−1). No effective discrimination between wild-type and any dilution below 100% mutant DNA was found.

FIG. 1C. The results of initial assessment of analytic sensitivity of Set3 primers (Tc=Tm−1). With use of manual mutation allele calling, the analytic sensitivity was found to be as little as 1%.

FIG. 1D. The results of initial assessment of analytic sensitivity of Set4 primers (Tc=Tm−1). The analytic sensitivity was found to be as little as 0.1%.

FIG. 2A. Finding the optimal conditions for COLD-PCR with primers Set4, testing condition 1 (Tc=80° C.; time of denature=3 sec). For these conditions, 1% mutant DNA corresponds to the difference value of around −4.

FIG. 2B. Finding the optimal conditions for COLD-PCR with primers Set4, testing condition 2 (Tc=80° C.; time of denature=10 sec). For these conditions, 1% mutant DNA corresponds to the difference value of around −3.

FIG. 2C. Finding the optimal conditions for COLD-PCR with primers Set4, testing condition 3 (Tc=79.5° C.; time of denature=3 sec). For these conditions, 1% mutant DNA corresponds to the difference value of around −5.

FIG. 2D. Finding the optimal conditions for COLD-PCR with primers Set4, testing condition 4 (Tc=79.5° C.; time of denature=10 sec). For these conditions, 1% mutant DNA corresponds to the difference value of around −3.

FIG. 2E. Finding the optimal conditions for COLD-PCR with primers Set4, testing condition 5 (Tc=79° C.; time of denature=3 sec). For these conditions, 1% mutant DNA corresponds to the difference value of around −7.

FIG. 3A. The results of testing primers and conditions for KRAS codon 12/13 mutation detection from Mancini et al. 2010. The dilution of 1% mutant DNA (G12S mutation) was detected with manual alleles call at difference of −3.

FIG. 3B. The results of testing primers and conditions for KRAS codon 12/13 mutation detection from Kristensen et al., 2010. The dilution of 1% mutant DNA was detected with manual alleles call at difference of −2.

FIG. 3C. The results of testing primers and conditions for KRAS codon 12/13 mutation detection from Carotenuto et al. 2011. The dilution of 1% mutant DNA was not detected, and the lowest dilution was 6% which we were able to detect.

FIG. 4A.—The results of initial assessment of analytic sensitivity of Set1 primers (Tc=Tm−1). The analytic sensitivity was found to be as little as 6.3%. Also the curves are difficult to interpret.

FIG. 4B.—The results of initial assessment of analytic sensitivity of Set2 primers (Tc=Tm−1). The analytic sensitivity was found to be as little as 1%.

FIG. 5.—Finding the optimal conditions for COLD-PCR with primer Set2, testing condition 1-3 (Tc=78.5, 78.0, 77.5° C.). Curves of different colour represent respective conditions: red, condition 1; blue, condition 2; green, condition 3. For condition 1 we see the highest deviation of curves from control, e.g. for lowest concentration of mutant DNA (EGFR ex19 del, PC9 cell line) the difference for red curve is about −5, while for blue and green curves it is about −4 and −3.5, respectively.

The Tc=78.5 (condition 1) provided the highest enrichment, so this Tc was chosen for subsequent experiments.

FIG. 6A.—The results of initial assessment of analytic sensitivity of Set1 primers (Tc=Tm−1). The sensitivity was found to be as little as 6.25% with manual allele calling.

FIG. 6B.—The results of initial assessment of analytic sensitivity of Set2 primers (Tc=Tm−1). The sensitivity was found to be as little as 1.6%

FIG. 7. Finding the optimal conditions for full COLD-PCR with primers Set2, testing condition 1-3 (Tc=86.5, 86.0, 85.5° C.).

### EXAMPLES

### Example 1—General Strategy for Primer Design

Overall, the following strategy was applied:

Custom primer sets were designed to encompass the region of interest taking into account some common rules (GC content 40%-60%, no self-complementarity, similar melting temperature, no secondary structures, etc.) and specific rules for COLD-PCR and HRM (product size <300, predicted difference in melting temperature for wild-type and mutant products).

The primer sets were tested to identify optimal conditions for effective discrimination between wild-type and mutant DNA. To do this, we experimentally identified the melting temperature (Tm) for the amplified sequences and used them as a starting point to find the critical temperature (Tc) for COLD-PCR which achieved the highest enrichment of mutant DNA (or analytic sensitivity, which we defined as a percentage of mutant DNA in a mixture of mutant/wild-type DNA).

The initial Tc was calculated as Tm−1. To identify the best Tc, we prepared serial dilutions of mutant DNA with 1% lowest concentration, ran COLD-PCR using different Tc values and then analysed the deviation of obtained melting curves from the wild-type control. The conditions under which the deviation of 1% DNA from the wild-type was the highest was considered the one to follow up. E.g. if under condition 1 the deviation was −3 units and under condition 2 the deviation was −5, then the condition 2 was chosen to follow up.

The most effective primers/conditions, were then compared with those publicly available.

### Example 2—Development of Optimised KRAS Primers and Conditions

4 sets of primers were designed according to Example 1.

Set1, 162 bp,
[SEQ ID NO: 22]
F-GGTCCTGCACCAGTAATATG;
[SEQ ID NO: 23]
R-GCCTGCTGAAAATGACTGAA
Set2, 170 bp,
[SEQ ID NO: 24]
F-AGAATGGTCCTGCACCAGTAA;
[SEQ ID NO: 25]
R-AAGGCCTGCTGAAAATGACT
Set3, 119 bp,
[SEQ ID NO: 26]
F-TTGTTGGATCATATTCGTCCAC;
[SEQ ID NO: 2]
R-AGGCCTGCTGAAAATGACTG
Set4, 138 bp,
[SEQ ID NO: 1]
F-AAAACAAGATTTACCTCTATTGTTGGA;
[SEQ ID NO: 2]
R-AGGCCTGCTGAAAATGACTG
(same as for Set3)

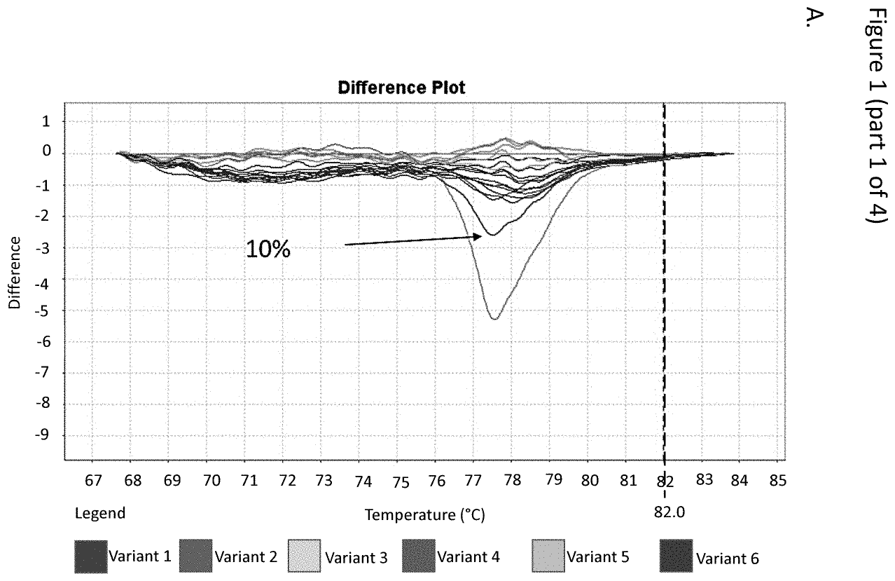

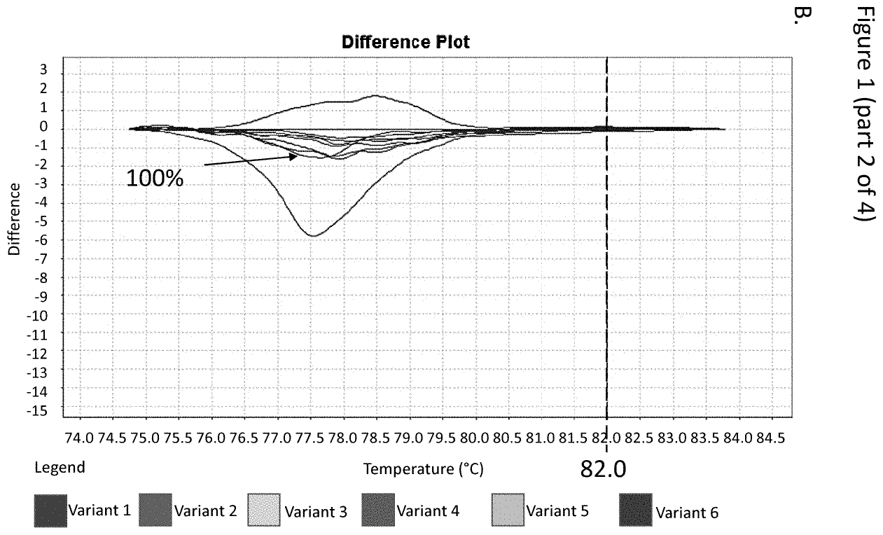

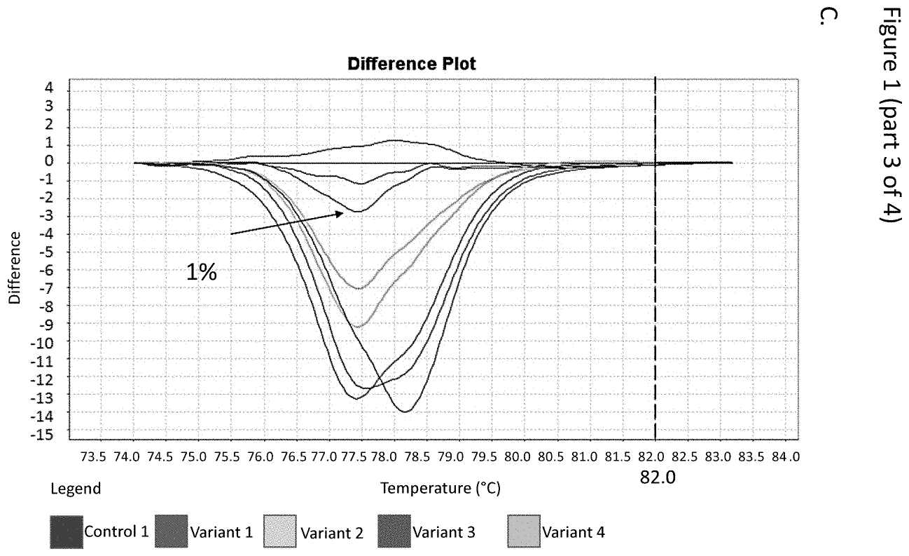

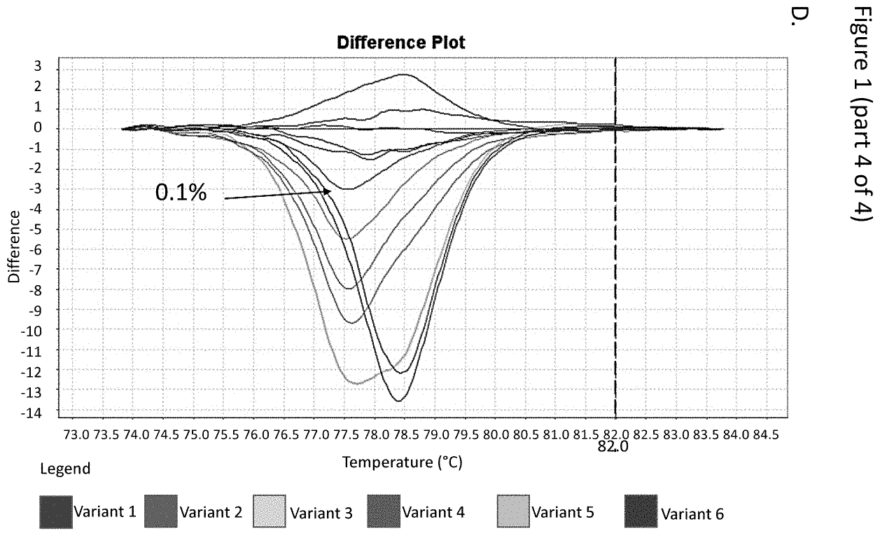

Difference plots were generated using a Tc of Tm−1. The results are shown in FIG. 1.

Set1 primers—The sensitivity was found to be as little as 10% mutant DNA.

Set2 primers—No effective discrimination between wild-type and any dilution below 100% mutant DNA was found.

Set3 primers—With use of manual mutation allele calling, the analytic sensitivity was found to be as little as 1%.

Set4 primers—The analytic sensitivity was found to be as little as 0.1%.

As the highest analytic sensitivity was found for the primers Set4, we then chose these primers to develop further and identify the optimal conditions to achieve the maximal enrichment of mutant DNA.

We tested 5 conditions taking into account Tc and time for denature under the Tc.

| Condition | Tc, ° C. | Time of denature, sec |
|---|---|---|
| 1 | 80 | 3 |
| 2 | 80 | 10 |
| 3 | 79.5 | 3 |
| 4 | 79.5 | 10 |
| 5 | 79 | 3 |

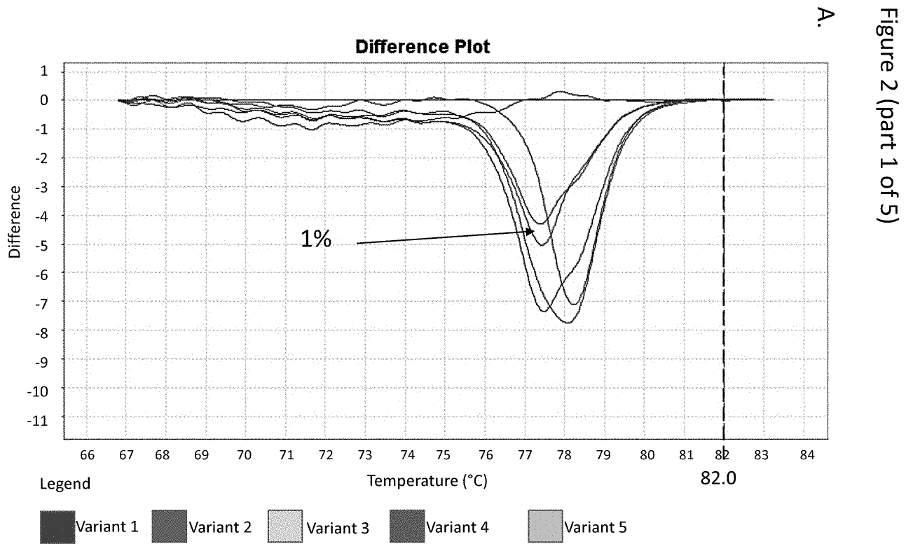

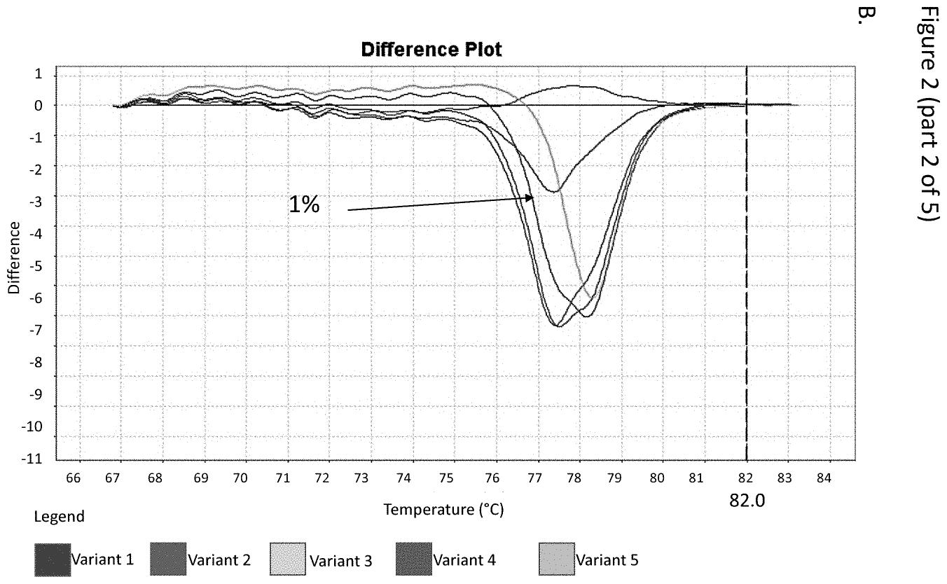

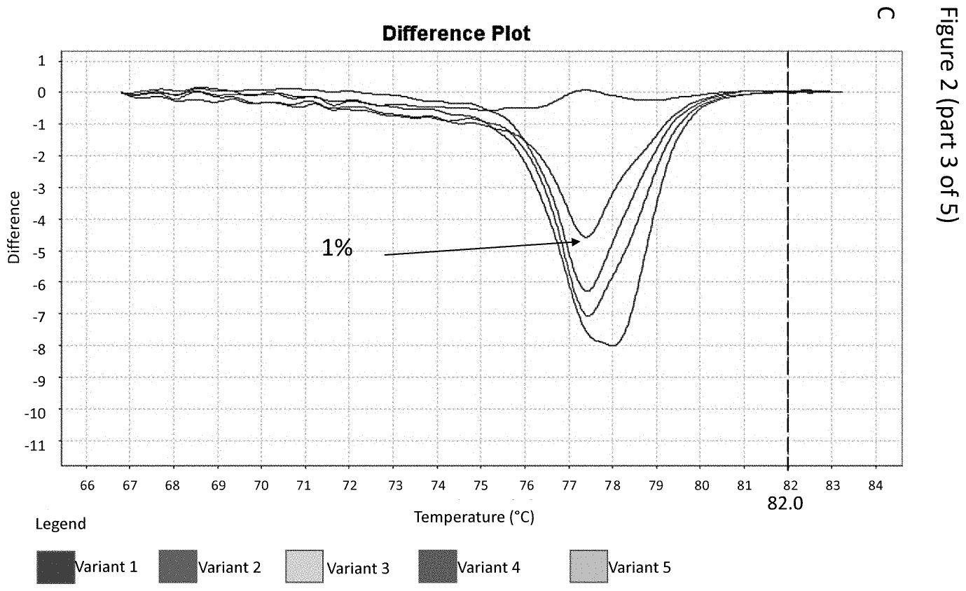

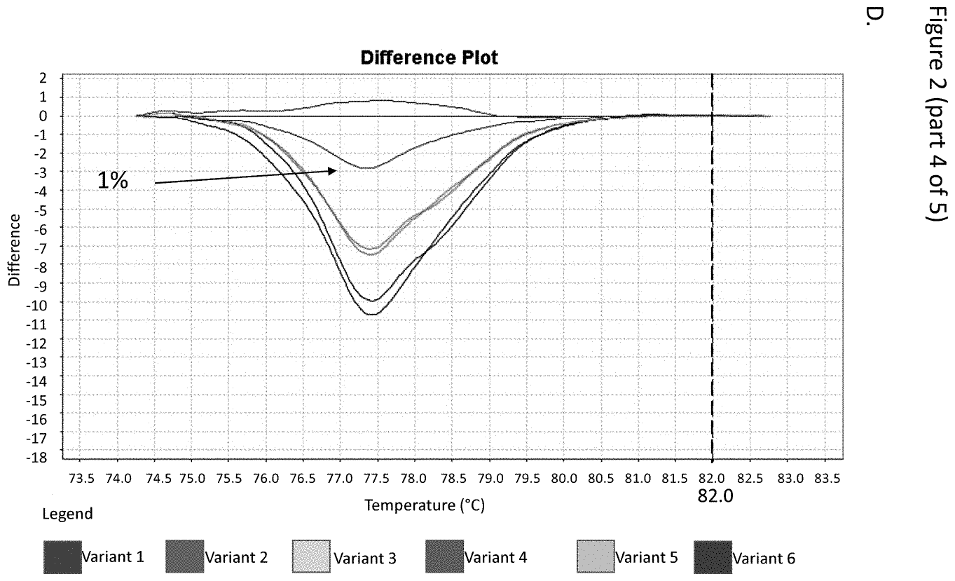

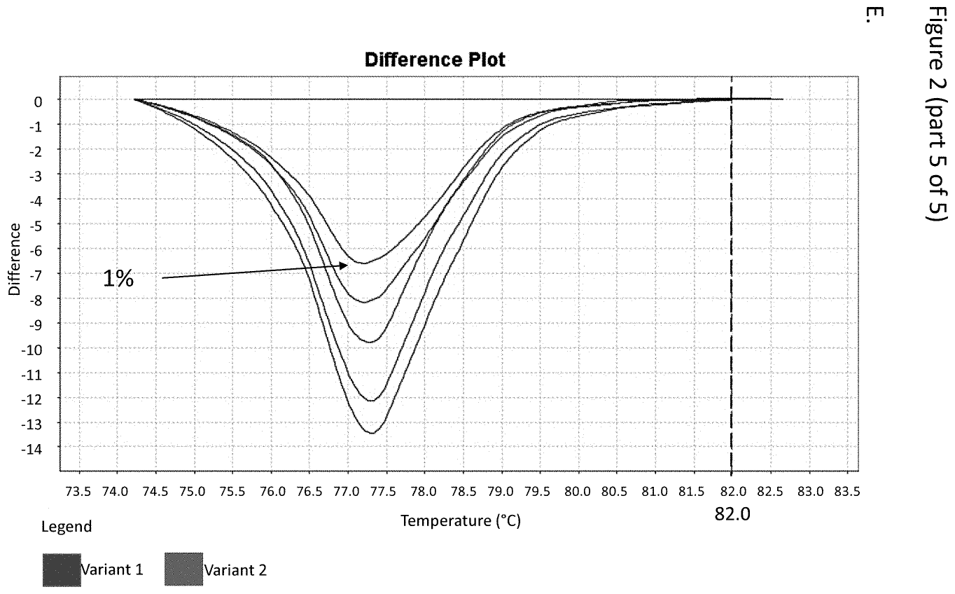

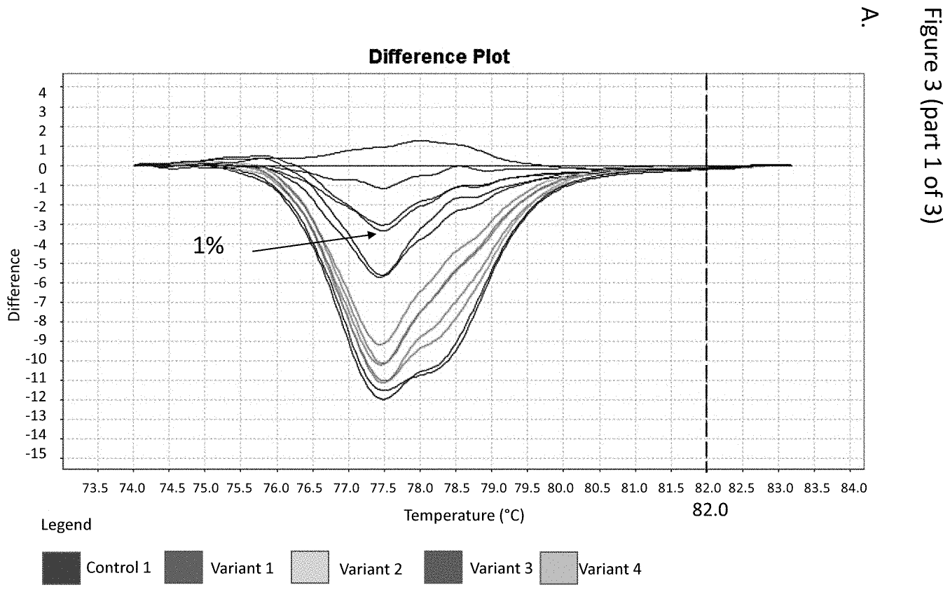

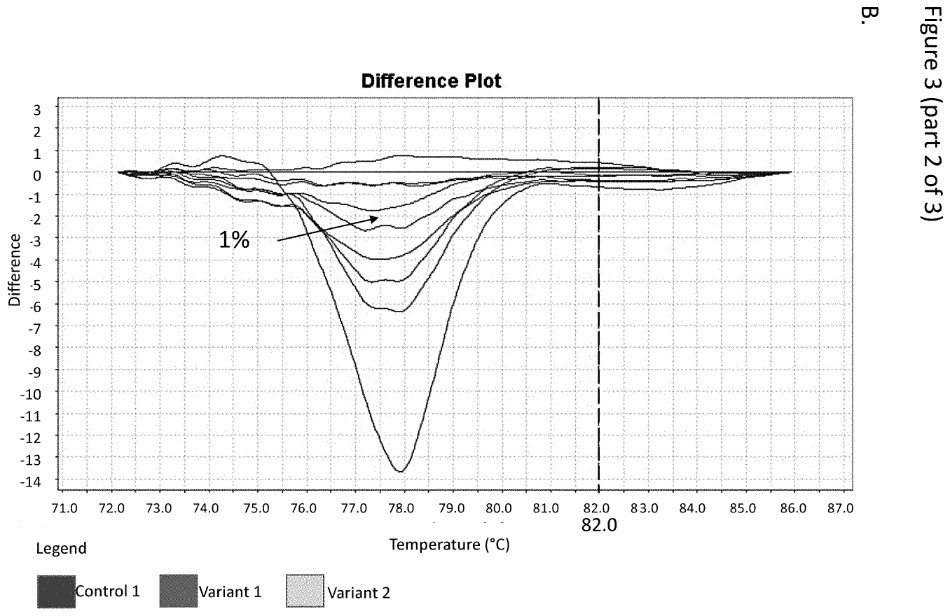

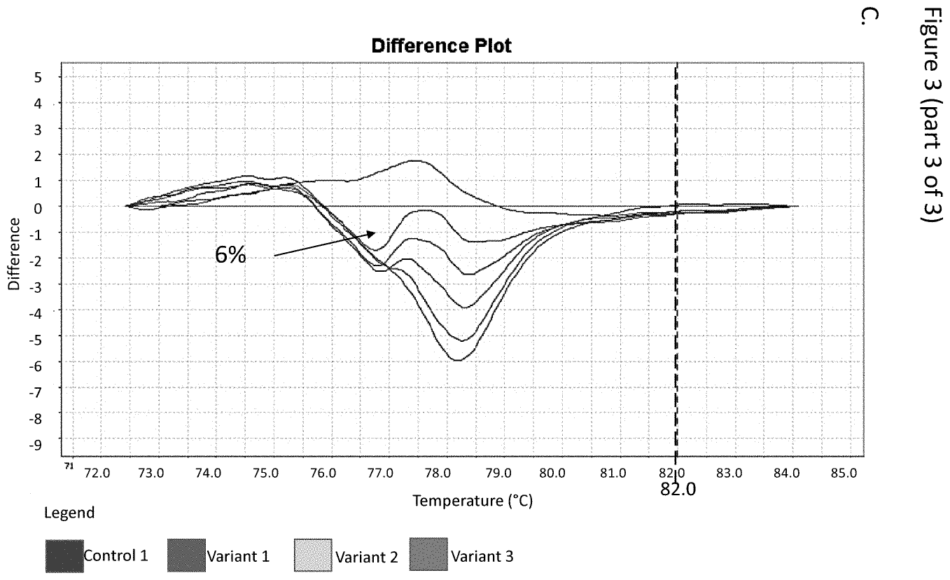

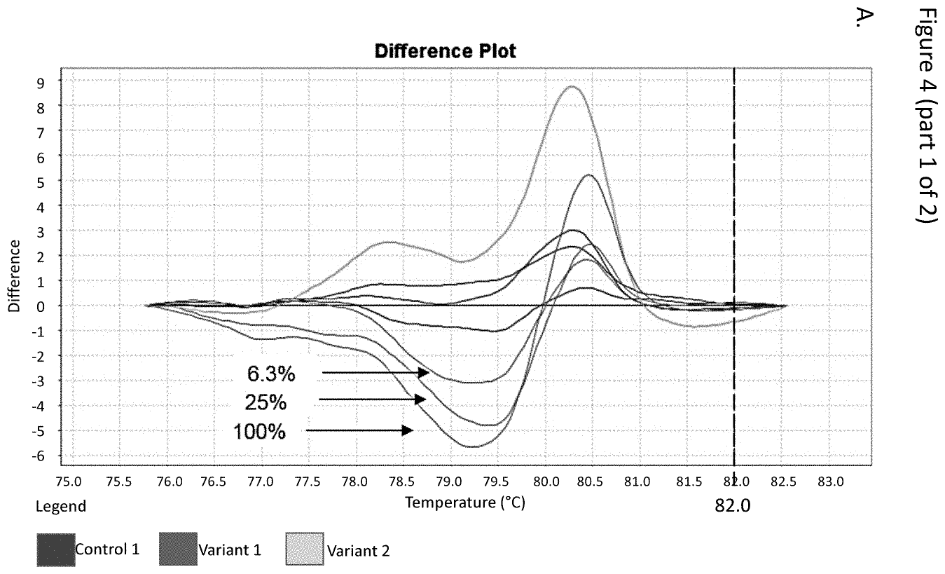

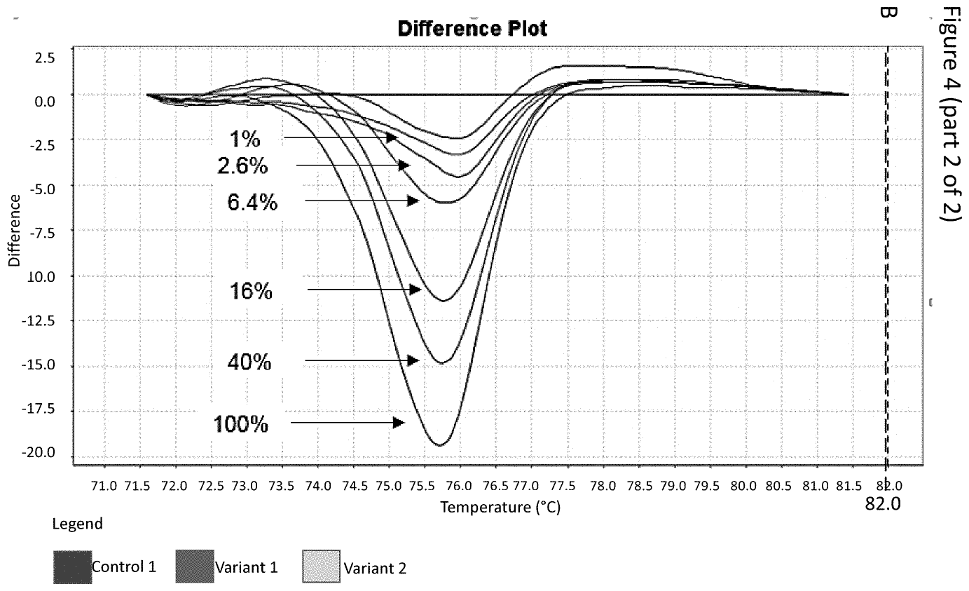

The results are shown in FIG. 2.

Condition 5 was found to show the highest deviation of a melting curve for 1% mutant DNA from control (the deviation of 1% mutant DNA from wild-type was −6.5). i.e. these conditions provide the highest enrichment. Therefore, we chose this primer set (SEQ ID NO: 1 and 2), and condition 5 (79° C. and 3 seconds) were chosen for subsequent analyses.

Thus, primers comprising SEQ ID NO: 1 and 2, are useful in the detection of mutations in the KRAS locus, particularly when used in combination with the reaction conditions of a Tc of 79° C. and even more particularly, but not essentially, with a denaturing time of 3 seconds.

### Example 3—Comparison of Optimised KRAS Primers and Conditions to the Prior Art

The primers and conditions developed above were compared to primers and conditions from the prior art, namely from three papers: Mancini et al. 2010; DOI: 10.2353/jmoldx.2010.100018; Kristensen et al., 2010; DOI 10.1002/humu.21358; and Carotenuto et al. 2011; DOI: 10.3892/ijo.2011.1221. The protocols described within the papers were followed with minor adjustments required to use equipment available in our lab (ABI 7500 fast instrument).

Mancini et al. 2010—The dilution of 1% mutant DNA was detected with manual alleles call at difference of −3.

Kristensen et al., 2010—The dilution of 1% mutant DNA was detected with manual alleles call at difference of −2.

Carotenuto et al. 2011—The dilution of 1% mutant DNA was not detected, and the lowest dilution was 6% which we were able to detect.

Thus, our primer set 4 (SEQ ID NO: 1 and 2) and respective conditions for fast COLD-PCR provide higher analytic sensitivity than competitors as we can detect 1% mutant DNA at difference −6.5 with automatic alleles call, while the best competitor can detect 1% mutant DNA at difference −3 with manual alleles call.

### Example 4—Comparison of Optimised KRAS Primers and Conditions to Prior Art Mutation Detection Kit

We carried out a comparison of our approach with a CE-IVD/US-IVD Cobas® KRAS mutation detection kit (Roche). We carried out the analysis of DNA extracted from FFPE blocks of 80 patients with primary or metastatic lung cancer. Using the Cobas® test, a total of 17 mutations was identified, while using our approach with primers of SEQ ID NO: 1 and 2, we found 2 more mutations (Table 1). These data testify that our approach is superior over a clinically approved Cobas® KRAS mutation detection kit.

TABLE 1
A comparison of COLD-PCR approach and cobas ® KRAS
mutation detection kit for identification of KRAS mutation
in tumour tissues of patients with lung cancer.

cobas ® KRAS
mutation detection
kit

| COLD-PCR | Positive | Negative |
|---|---|---|
| Positive | 17 | 2 |
| Negative | 0 | 61 |

### Example 5—the Optimised KRAS Primers and Conditions are Suitable for Use with ctDNA Giving Superior Mutation Detection than when Used with Tumour Samples

We also investigated whether our assays can detect mutations in low quality DNA, such as ctDNA extracted from plasma. If this is possible, it would provide a much more simple and low cost way to screen subjects for mutations than having to extract DNA from tumour biopsies.

To do so, we carried out the analysis of mutations in tumours and matched ctDNA specimens of 82 lung cancer patients.

First, we found a very good concordance between ctDNA and tumours (Table 2), with 18 out of 19 mutations in tumours found in ctDNA (94.7%). Also, we found more KRAS mutations in ctDNA than in matched tumours, thus suggesting that the analysis of ctDNA can be used as a sensitive and specific test for KRAS mutations in lung cancer patients.

TABLE 2
Breakdown and statistics of concordance between mutation
detection in DNA obtained from tumours and blood-derived DNA

Tumours,
COLD-PCR/
HRM assay

| ctDNA | Positive | Negative |
|---|---|---|
| Positive | 18 | 7 |
| Negative | 1 | 56 |

| | |
|---|---|
| Sensitivity (95% CI) | 0.947 (0.774-0.999) |
| Specificity (95% CI) | 0.889 (0.801-0.954) |

### Example 6—Development of Optimised EGFR Primers and Conditions

Ex19

3 sets of primers were designed according to Example 1.

Set1, 210 bp;
[SEQ ID NO: 27]
F-GCTGGTAACATCCACCCAGA;
[SEQ ID NO: 28]
R-CCACACAGCAAAGCAGAAAC
Set2, 101 bp;
[SEQ ID NO: 3]
F-GTTAAAATTCCCGTCGCTATCA;
[SEQ ID NO: 4]
R-GACCCCCACACAGCAAA
Set3, 103 bp;
[SEQ ID NO: 5]
F-AAGTTAAAATTCCCGTCGCTATC;
[SEQ ID NO: 4]
R-GACCCCCACACAGCAAA

The Tc in both instances was Tm−1.

Set1 primers—The analytic sensitivity was found to be as little as 6.3%. Also the curves are difficult to interpret.

Set2 primers—The analytic sensitivity was found to be as little as 1%.

The results for Set2 and Set3 primers were the same, so all subsequent experiments were carried out with Set2 primers. However, Set 3 primers are also considered to be useful primers of the invention.

We then tested 3 conditions taking into account Tc.

| Condition | Tc, ° C. |
|---|---|
| 1 | 78.5 |
| 2 | 78.0 |
| 3 | 77.5 |

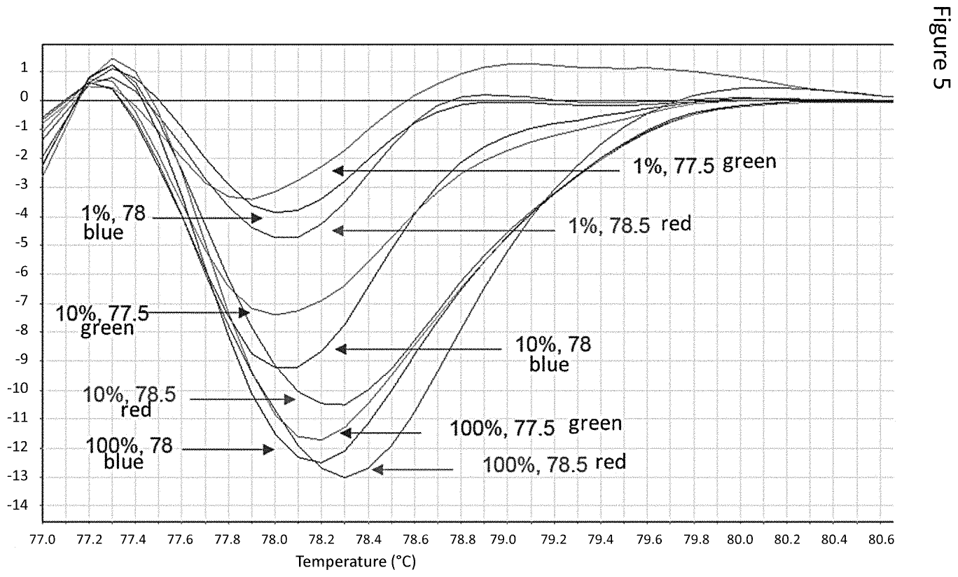

The results are shown in FIG. 5.

The Tc=78.5 provided the highest enrichment, so this Tc was chosen for subsequent experiments.

Ex21 (L858R)

2 sets of primers were designed according to Example 1.

Set1, 180 bp,
[SEQ ID NO: 29]
F-TTCCCATGATGATCTGTCCC,
[SEQ ID NO: 30]
R-TCTTTCTCTTCCGCACCCAG
Set2, 80 bp,
[SEQ ID NO: 6]
F-GCAGCATGTCAAGATCACAGA,
[SEQ ID NO: 7]
R-TGCCTCCTTCTGCATGGTAT

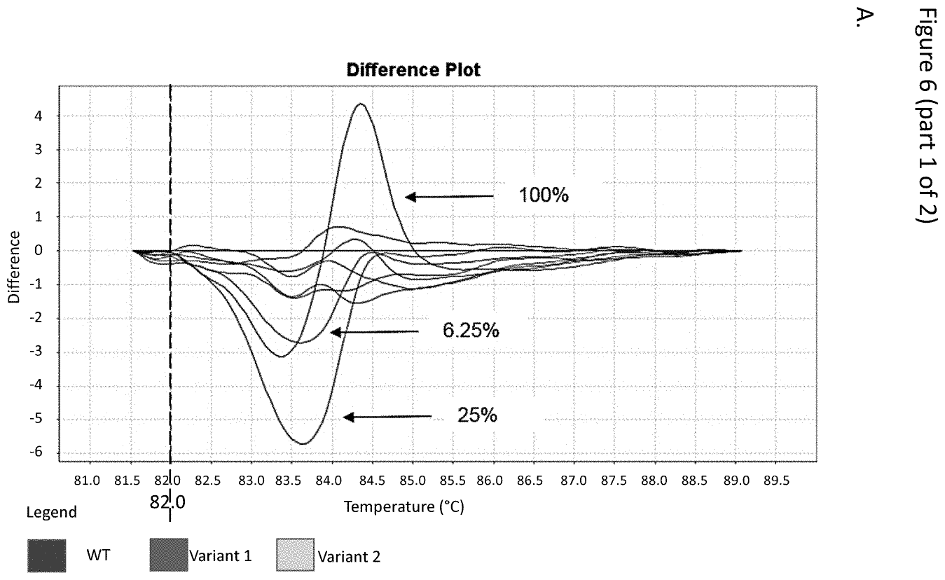

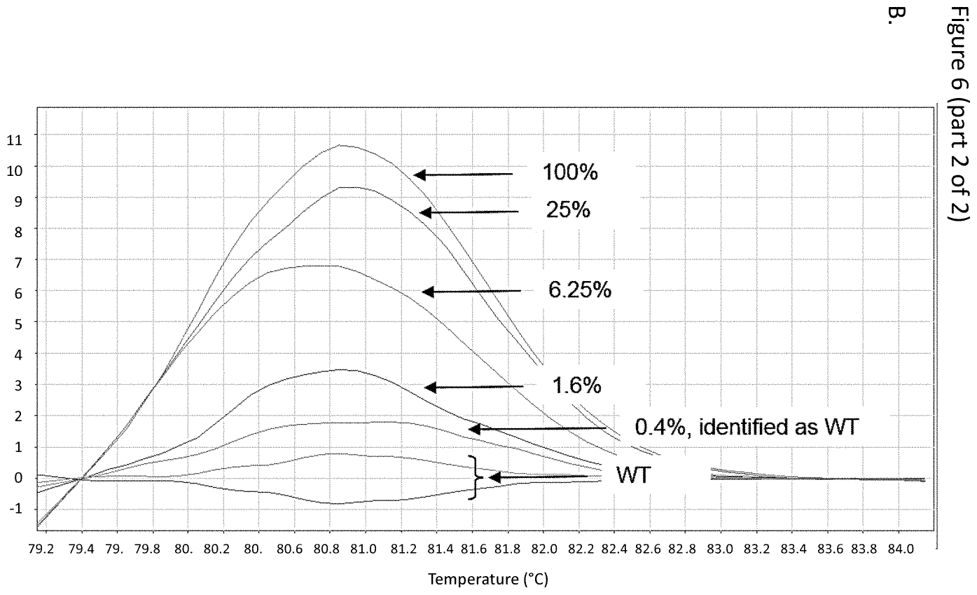

As L858R is a Tm gaining mutation, full COLD-PCR was used. Results are shown in FIG. 6. Tc was Tm−1 in both cases.

Set1 primers—The sensitivity was found to be as little as 6.25% with manual allele calling.

Set2 primers—The sensitivity was found to be as little as 1.6%

We then tested 3 conditions taking into account Tc.

| Condition | Tc, ° C. |
|---|---|
| 1 | 86.5 |
| 2 | 86.0 |
| 3 | 85.5 |

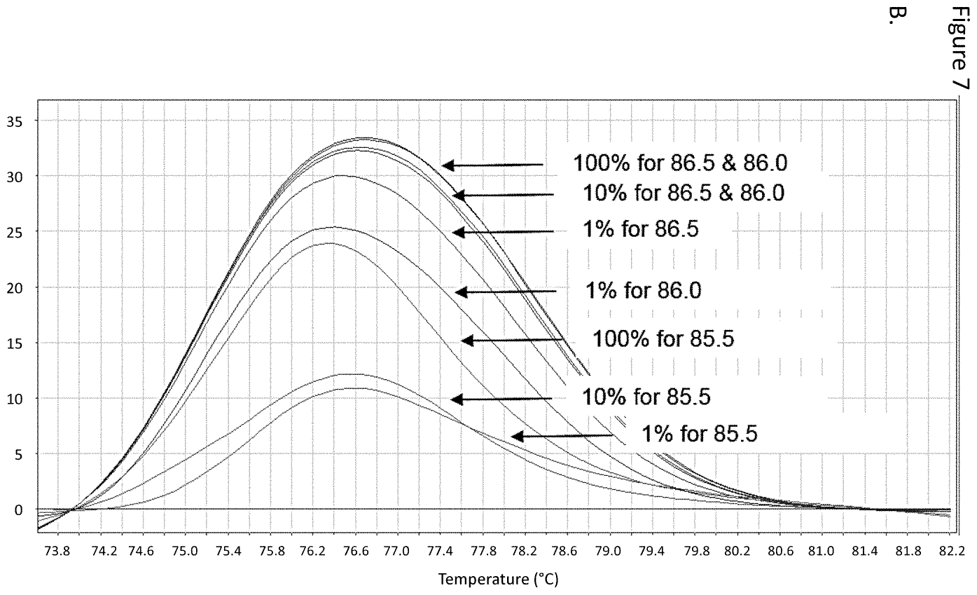

Results are shown in FIG. 7. Tc=86.5 was found to be optimal.

### Example 7—Comparison of Optimised EGFR Primers and Conditions to Prior Art Mutation Detection Kit

We compared the performance of our assays versus Cobas® EGFR tests (Roche) in a cohort of 95 lung cancer patients, using paraffin embedded tissue samples. Using the Cobas® tests we have found 4 Ex19 deletions, while with the COLD-PCR assay we additionally identified 8 mutations (Table 3). Absolute concordance between the tests was found for Ex21 L858R mutations (Table 4).

TABLE 3
A comparison of COLD-PCR approach and cobas ® EGFR
mutation detection kit for identification of EGFR Ex19
deletions in tumour tissues of patients with lung cancer

cobas ® EGFR
mutation detection
kit (Ex19)

| COLD-PCR | Positive | Negative |
|---|---|---|
| Positive | 4 | 8 |
| Negative | 0 | 83 |

TABLE 4
A comparison of COLD-PCR approach and cobas ® EGFR
mutation detection kit for identification of EGFR Ex19 deletions
in tumour tissues of patients with lung cancer

cobas ® EGFR
mutation detection
kit (Ex19)

| COLD-PCR | Positive | Negative |
|---|---|---|
| Positive | 3 | 0 |
| Negative | 0 | 92 |

Thus, our COLD-PCR assays outperforms or are equivalent to cobas EGFR mutation detection kit.

We also tested the performance of our COLD-PCR approach for ctDNA. Tables 5 and 6 provide the results of a comparison of mutation detection in ctDNA as compared with matched FFPE tumours for EGFR Ex19 and L858R (Ex21) mutations in lung cancer patients.

TABLE 5
A comparison of the performance of mutation detection in EGFR
Ex19 in ctDNA and match FFPE DNA in lung cancer patients
using COLD-PCR approach

FFPE

| ctDNA | Positive | Negative |
|---|---|---|
| Positive | 6 | 3 |
| Negative | 2 | 38 |

TABLE 6
A comparison of the performance of L858R mutation detection in
EGFR Ex21 in ctDNA and match FFPE DNA in lung cancer patients
using COLD-PCR approach

FFPE

| ctDNA | Positive | Negative |
|---|---|---|
| Positive | 2 | 0 |
| Negative | 1 | 92 |

Thus, both Ex19 deletions and L585R mutation in EGFR gene can effectively be detected in ctDNA using developed COLD-PCR approach. Again, we can see that some mutations discovered in ctDNA are not detected in FFPE.

### Example 8—Optimised Primers for Detection of Various p53 Mutations

Using the same methodology, we developed primers and conditions for COLD-PCR assessment of mutations. Taken into account, that in the case of TP53, no specific mutations are of interest, the whole sequence of exons 5 to 8 (account for 95% of all TP53 mutations) were analysed. Exon 5 was split into 3 fragments and exon 8 was split into 2 fragments due to their length. Currently, there is no clinically accepted commercial test for TP53 mutations, therefore we only compared the performance of mutations detection in ctDNA and FFPE.

A good concordance between ctDNA and FFPE for 92 lung cancer patients was seen for all exons, with most mutations detected in Exon 5 (table 7).

TABLE 7
A comparison of the performance of mutation detection in TP53 gene
in ctDNA and match FFPE DNA in lung cancer patients using
COLD-PCR approach

| | |
|---|---|
| TP53 | FFPE |

| exon | ctDNA | Positive | Negative |
|---|---|---|---|
| Ex 5 | Positive | 40 | 10 |
| | Negative | 3 | 39 |
| Ex 6 | Positive | 9 | 7 |
| | Negative | 3 | 73 |
| Ex 7 | Positive | 9 | 3 |
| | Negative | 3 | 77 |
| Ex 8 | Positive | 7 | 8 |
| | Negative | 3 | 74 |

The optimised primers are shown below in Table 5—

TABLE 5

| | | Critical | |
|---|---|---|---|
| | | temperature | |
| | | (Tc) for COLD- | Time at |
| Exon | Sequence, 5′-3′ | PCR, ° C. | Tc, sec |
| 5-1 | AACCAGCCCTGTCGTCTCT | 86.7 | 10 |
| | [SEQ ID NO: 8] | | |
| | CAAGCAGTCACAGCACATGA | | |
| | [SEQ ID NO: 9] | | |
| 5-2 | CTGAGCAGCGCTCATGGT | 89.0 | 10 |
| | [SEQ ID NO: 10] | | |
| | GTGCAGCTGTGGGTTGATTC | | |
| | [SEQ ID NO: 11] | | |
| 5-3 | GGGGGTGTGGAATCAAC | 83.0 | 10 |
| | [SEQ ID NO: 12] | | |
| | ACTTGTGCCCTGACTTTCAA | | |
| | [SEQ ID NO: 13] | | |
| 6 | CAGTTGCAAACCAGACCTCA | 87.5 | 3 |
| | [SEQ ID NO: 14] | | |
| | GGCCTCTGATTCCTCACTGAT | | |
| | [SEQ ID NO: 15] | | |
| 7 | GGCTCCTGACCTGGAGTCTT | 83.5 | 10 |
| | [SEQ ID NO: 16] | | |
| | CTTGGGCCTGTGTTATCTCC | | |
| | [SEQ ID NO: 17] | | |
| 8-1 | TCTTGCGGAGATTCTCTTCC | 81.8 | 20 |
| | [SEQ ID NO: 18] | | |
| | GCCTCTTGCTTCTCTTTTCCT | | |
| | [SEQ ID NO: 19] | | |
| 8-2 | GCTTCTTGTCCTGCTTGCTT | 85.3 | 10 |
| | [SEQ ID NO: 20] | | |
| | CTACTGGGACGGAACAGCTT | | |
| | [SEQ ID NO: 21] | | |

### Example 9—Detailed Protocol for the Detection of Mutations in the KRAS Codon 12/13 Locus

The below is a detailed protocol for the detection of the mutations in the KRAS codon 12/13 locus. It will be appreciated that not every step needs to be followed precisely, not even included, and the precise amounts of and types of reagent may vary without affecting the outcome, all of which the skilled person will be well aware.

Equipment

PCR-workstation

QIAGEN RotorGene real-time PCR instrument

Dedicated PCR pipettes

Materials

Type-it HRM PCR Kit (QIAGEN)

PCR-grade water (supplied with the kit or equivalent)

RotorGene 0.1 uL PCR strips

Control DNA

Notes:
All pre-PCR steps must be carried out in a PCR-workstation
Use dedicated PCR pipettes and sterile filter tips
Equilibrate concentrations of the DNA before use, so all the samples were at same concentration; variation ˜5% is acceptable.

Procedure 1 (COLD-PCR):
Prepare spreadsheet with samples allocated in PCR instrument rotor.
Completely thaw oligonucleotides for COLD-PCR and reagents at RT before use; shake or mix them well and spin down.
Calculate the amount of reagents required for the number of reactions (including control DNA)+5%. For every sample, including control DNA, set up at least 2 technical replicates.
Prepare master mix (MM) in a 1.5-2.0 mL sterile tube:

| | Amount per 1 sample, | |
|---|---|---|
| Reagent | ul | Final concentration |
| Type-it HRM mix, 2x | 12.5 | 1x |
| Primer Fwd, 10 uM | 1.75 | 700 nM |
| Primer Rev, 10 uM | 1.75 | 700 nM |
| Water | 8 | — |
| DNA | 1 | — |

### Primers Sequence:

| [SEQ ID NO: 1] | |
|---|---|
| Fwd: | 5′-AAAACAAGATTTACCTCTATTGTTGGA-3′ |
| [SEQ ID NO: 2] | |
| Rev: | 5′-AGGCCTGCTGAAAATGACTG-3′ |

Aliquot 24 uL of MM into PCR-tubes
Put 1 uL DNA for each sample into the tubes according to the sample allocations.
Close the tubes with caps and place them into rotor of the RotorGene; close the RotorGene lid.
Turn on RotorGene and attached laptop; set up program for fast or full COLD-PCR using the following profile:

Fast COLD-PCR

| Stage | Temp | Time | Cycles |
|---|---|---|---|
| Activation | 95° C. | 5 min | 1 |
| Pre-PCR | 95° C. | 10 sec | 20 |
| | 55° C. | 30 sec | |
| | 72° C. | 10 sec | |
| COLD | 79° C. | 3 sec | 40/45 |
| | 55° C. | 30 sec | |
| | 72° C. | 10 sec | |
| HRM | 68-90 | | 1 |

Full COLD-PCR

| Stage | Temp | Time | Cycles |
|---|---|---|---|
| Activation | 95° C. | 5 min | 1 |
| Pre-PCR | 95° C. | 10 sec | 20 |
| | 60° C. | 1 min | |
| COLD | 95° C. | 10 sec | 40/45 |
| | 70° C. | 5 min | |
| | 79.5° C. | 3 sec | |
| | 55° C. | 30 sec | |
| | 72° C. | 10 sec | |
| HRM | 68-90 | | 1 |

Run the program
Analyze the results using RotorGene software; mutations will be detected automatically, bust manual adjustment may require.

### Example 10—Detailed Protocol for the Detection of Mutations in the EGFR Exon 19 and Exon 21

The below is a detailed protocol for the detection of the mutations in the EGFR exon 19 and exon 21. It will be appreciated that not every step need to be followed precisely, not even included, and the precise amounts of and types of reagent may vary without affecting the outcome, all of which the skilled person will be well aware.

Equipment

PCR-workstation

QIAGEN RotorGene real-time PCR instrument

Dedicated PCR pipettes

Materials

Type-it HRM PCR Kit (QIAGEN)

PCR-grade water (supplied with the kit or equivalent)

RotorGene 0.1 uL PCR strips

Control DNA

Notes:
All pre-PCR steps must be carried out in a PCR-workstation
Use dedicated PCR pipettes and sterile filter tips
Equilibrate concentrations of the DNA before use, so all the samples were at same concentration; variation ˜5% is acceptable.
Set up Ex19 and Ex21 reactions separately, no multiplexing
Ex21 L858R mutation is a temperature gaining, this must be taken into account during interpretation of the results

Sequences of Oligos:

| EGFR Ex19 | |
|---|---|
| Fwd: | 5′-GTTAAAATTCCCGTCGCTATCA |
| Rev: | 5′-GACCCCCACACAGCAAA |
| EGFR Ex21 | |
| Fwd: | 5′-GCAGCATGTCAAGATCACAGA |
| Rev: | 5′-TGCCTCCTTCTGCATGGTAT |

Procedure 1 (COLD-PCR):
Prepare spreadsheet with samples allocated in PCR instrument rotor.
Completely thaw oligonucleotides for COLD-PCR and reagents at RT before use; shake or mix them well and spin down.
Calculate the amount of reagents required for the number of reactions (including control DNA)+5%. For every sample, including control DNA, set up at least 2 technical replicates.
Prepare master mix (MM) in a 1.5-2.0 mL sterile tube:

| | Amount per 1 sample, | |
|---|---|---|
| Reagent | ul | Final concentration |
| Type-it HRM mix, 2x | 12.5 | 1x |
| Primer Fwd, 10 uM | 1.75 | 700 nM |
| Primer Rev, 10 uM | 1.75 | 700 nM |
| Water | 8 | — |
| DNA | 1 | — |

Aliquot 24 uL of MM into PCR-tubes
Put 1 uL DNA for each sample into the tubes according to the sample allocations.
Close the tubes with caps and place them into rotor of the RotorGene; close the RotorGene lid.
Turn on RotorGene and attached laptop; set up program for fast or full COLD-PCR using the following profiles:

Ex19 Fast COLD-PCR

| Stage | Temp | Time | Cycles |
|---|---|---|---|
| Activation | 95° C. | 5 min | 1 |
| Pre-PCR | 95° C. | 10 sec | 20 |
| | 55° C. | 30 sec | |
| | 72° C. | 10 sec | |
| COLD | 78.5° C. | 3 sec | 40/45 |
| | 55° C. | 30 sec | |
| | 72° C. | 10 sec | |
| HRM | 68-90 | | 1 |

Ex21 Full COLD-PCR

| Stage | Temp | Time | Cycles |
|---|---|---|---|
| Activation | 95° C. | 5 min | 1 |
| Pre-PCR | 95° C. | 10 sec | 20 |
| | 60° C. | 1 min | |
| COLD | 95° C. | 10 sec | 40/45 |
| | 70° C. | 5 min | |
| | 86° C. | 3 sec | |
| | 55° C. | 30 sec | |
| | 72° C. | 10 sec | |
| HRM | 68-90 | | 1 |

Run the program

Analyse the results using RotorGene software; mutations will be detected automatically, but manual adjustment may be required.

### Example 11—Detailed Protocol for the Detection of Mutations in Various Regions of p53

The below is a detailed protocol for the detection of the mutations in various regions of p53. It will be appreciated that not every step need to be followed precisely, not even included, and the precise amounts of and types of reagent may vary without affecting the outcome, all of which the skilled person will be well aware.

SOP Mutation detection in exons 5-8 of TP53 gene

Equipment

PCR-workstation

QIAGEN RotorGene real-time PCR instrument

Dedicated PCR pipettes

Materials

Type-it HRM PCR Kit (QIAGEN)

PCR-grade water (supplied with the kit or equivalent)

RotorGene 0.1 uL PCR strips

Control DNA

Notes:
All pre-PCR steps must be carried out in a PCR-workstation
Use dedicated PCR pipettes and sterile filter tips
Equilibrate concentrations of the DNA before use, so all the samples were at same concentration; variation ˜5% is acceptable.

Sequences of Primers:

| | | Critical | |
|---|---|---|---|
| | | temperature | |
| | | (Tc) for COLD- | Time at |
| Exon | Sequence, 5′-3′ | PCR, ° C. | Tc, sec |
| 5-1 | AACCAGCCCTGTCGTCTCT | 86.7 | 10 |
| | [SEQ ID NO: 8] | | |
| | CAAGCAGTCACAGCACATGA | | |
| | [SEQ ID NO: 9] | | |
| 5-2 | CTGAGCAGCGCTCATGGT | 89.0 | 10 |
| | [SEQ ID NO: 10] | | |
| | GTGCAGCTGTGGGTTGATTC | | |
| | [SEQ ID NO: 11] | | |
| 5-3 | GGGGGTGTGGAATCAAC | 83.0 | 10 |
| | [SEQ ID NO: 12] | | |
| | ACTTGTGCCCTGACTTTCAA | | |
| | [SEQ ID NO: 13] | | |
| 6 | CAGTTGCAAACCAGACCTCA | 87.5 | 3 |
| | [SEQ ID NO: 14] | | |
| | GGCCTCTGATTCCTCACTGAT | | |
| | [SEQ ID NO: 15] | | |
| 7 | GGCTCCTGACCTGGAGTCTT | 83.5 | 10 |
| | [SEQ ID NO: 15] | | |
| | CTTGGGCCTGTGTTATCTCC | | |
| | [SEQ ID NO: 17] | | |
| 8-1 | TCTTGCGGAGATTCTCTTCC | 81.8 | 20 |
| | [SEQ ID NO: 18] | | |
| | GCCTCTTGCTTCTCTTTTCCT | | |
| | [SEQ ID NO: 19] | | |
| 8-2 | GCTTCTTGTCCTGCTTGCTT | 85.3 | 10 |
| | [SEQ ID NO: 20] | | |
| | CTACTGGGACGGAACAGCTT | | |
| | [SEQ ID NO: 21] | | |

Procedure (COLD-PCR):
Prepare spreadsheet with samples allocated in PCR instrument rotor.
Completely thaw oligonucleotides for COLD-PCR and reagents at RT before use; shake or mix them well and spin down.
Calculate the amount of reagents required for the number of reactions (including control DNA)+5%. For every sample, including control DNA, set up at least 2 technical replicates.
Prepare master mix (MM) in a 1.5-2.0 mL sterile tube:

| | Amount per 1 sample, | |
|---|---|---|
| Reagent | ul | Final concentration |
| Type-it HRM mix, 2x | 12.5 | 1x |
| Primer Fwd, 10 uM | 1.75 | 700 nM |
| Primer Rev, 10 uM | 1.75 | 700 nM |
| Water | 8 | — |
| DNA | 1 | — |

Aliquot 24 uL of MM into PCR-tubes
Put 1 uL DNA for each sample into the tubes according to the sample allocations.
Close the tubes with caps and place them into rotor of the RotorGene; close the RotorGene lid.
Turn on RotorGene and attached laptop; set up program for fast COLD-PCR using the following profile:

Fast COLD-PCR

| Stage | Temp | Time | | Cycles |
|---|---|---|---|---|
| Activation | 95° C. | 5 | min | 1 |
| Pre-PCR | 95° C. | 10 | sec | 20 |
| | 55° C. | 30 | sec | |
| | 72° C. | 10 | sec | |
| COLD | Tc° C.* | 3-20 | sec* | 40/45 |
| | 55° C. | 30 | sec | |
| | 72° C. | 10 | sec | |
| HRM | 68-90 | | | 1 |

*see table above for details

Run the program
Analyse the results using RotorGene software; mutations will be detected automatically, but manual adjustment may be required.

1. A chemically synthesized nucleic acid of less than 50 nucleotides in length comprising any one of the following sequences:
[SEQ ID NO: 1]
AAAACAAGATTTACCTCTATTGTTGGA
[SEQ ID NO: 2]
AGGCCTGCTGAAAATGACTG
[SEQ ID NO: 3]
GTTAAAATTCCCGTCGCTATCA
[SEQ ID NO: 4]
GACCCCCACACAGCAAA
[SEQ ID NO: 5]
AAGTTAAAATTCCCGTCGCTATC
[SEQ ID NO: 6]
GCAGCATGTCAAGATCACAGA
[SEQ ID NO: 7]
TGCCTCCTTCTGCATGGTAT
[SEQ ID NO: 8]
AACCAGCCCTGTCGTCTCT
[SEQ ID NO: 9]
CAAGCAGTCACAGCACATGA
[SEQ ID NO: 10]
CTGAGCAGCGCTCATGGT
[SEQ ID NO: 11]
GTGCAGCTGTGGGTTGATTC
[SEQ ID NO: 12]
GGGGGTGTGGAATCAAC
[SEQ ID NO: 13]
ACTTGTGCCCTGACTTTCAA
[SEQ ID NO: 14]
CAGTTGCAAACCAGACCTCA
[SEQ ID NO: 15]
GGCCTCTGATTCCTCACTGAT
[SEQ ID NO: 16]
GGCTCCTGACCTGGAGTCTT
[SEQ ID NO: 17]
CTTGGGCCTGTGTTATCTCC
[SEQ ID NO: 18]
TCTTGCGGAGATTCTCTTCC
[SEQ ID NO: 19]
GCCTCTTGCTTCTCTTTTCCT
[SEQ ID NO: 20]
GCTTCTTGTCCTGCTTGCTT
[SEQ ID NO: 21]
CTACTGGGACGGAACAGCTT;
or a sequence with greater than 80% homology to any one of the above sequences.

2. A method for detecting the presence of a mutation in the KRAS gene, and/or the p53 gene, and/or the EGFR gene in a sample obtained from a subject, the method comprising amplification of the region of interest using any two of the nucleic acids of claim 1, wherein:
[SEQ ID NO: 1]
AAAACAAGATTTACCTCTATTGTTGGA
and
[SEQ ID NO: 2]
AGGCCTGCTGAAAATGACTG
are used to amplify the KRAS locus;
[SEQ ID NO: 3]
GTTAAAATTCCCGTCGCTATCA
and
[SEQ ID NO: 4]
GACCCCCACACAGCAAA
are used to amplify the EGFR exon 19 locus;
[SEQ ID NO: 5]
AAGTTAAAATTCCCGTCGCTATC
and
[SEQ ID NO: 4]
GACCCCCACACAGCAAA
are used to amplify the EGFR exon 19 locus;
[SEQ ID NO: 6]
GCAGCATGTCAAGATCACAGA
and
[SEQ ID NO: 7]
TGCCTCCTTCTGCATGGTAT
are used to amplify the EGFR exon 21 locus;
[SEQ ID NO: 8]
AACCAGCCCTGTCGTCTCT
and
[SEQ ID NO: 9]
CAAGCAGTCACAGCACATGA
are used to amplify the p53 exon 5-1 locus;
[SEQ ID NO: 10]
CTGAGCAGCGCTCATGGT
and
[SEQ ID NO: 11]
GTGCAGCTGTGGGTTGATTC
are used to amplify the p53 exon 5-2 locus;
[SEQ ID NO: 12]
GGGGGTGTGGAATCAAC
and
[SEQ ID NO: 13]
ACTTGTGCCCTGACTTTCAA
are used to amplify the p53 exon 5-3 locus;
[SEQ ID NO: 14]
CAGTTGCAAACCAGACCTCA
and
[SEQ ID NO: 15]
GGCCTCTGATTCCTCACTGAT
are used to amplify the p53 exon 6 locus;
[SEQ ID NO: 16]
GGCTCCTGACCTGGAGTCTT
and
[SEQ ID NO: 17]
CTTGGGCCTGTGTTATCTCC
are used to amplify the p53 exon 7 locus;
[SEQ ID NO: 18]
TCTTGCGGAGATTCTCTTCC
and
[SEQ ID NO: 19]
GCCTCTTGCTTCTCTTTTCCT
are used to amplify the p53 exon 8-1 locus; and
[SEQ ID NO: 20]
GCTTCTTGTCCTGCTTGCTT
and
[SEQ ID NO: 21]
CTACTGGGACGGAACAGCTT
are used to amplify the p53 exon 8-2 locus.

3. The method of claim 2 wherein the amplification is performed by PCR, optionally wherein the PCR is COLD-PCR.

4. The method of claim 3 wherein:
where the target is KRAS and the nucleic acids are AAAACAAGATTTACCTCTATTGTTGGA [SEQ ID NO:1] and AGGCCTGCTGAAAATGACTG [SEQ ID NO: 2], the Tc of the COLD-PCR is 79° C., optionally wherein the denaturing time is 3 seconds;
where the target is EGFR and the nucleic acids are GTTAAAATTCCCGTCGCTATCA [SEQ ID NO: 3] and GACCCCCACACAGCAAA [SEQ ID NO: 4], the Tc of the COLD-PCR is 78.5° C.;
where the target is EGFR and the nucleic acids are AAGTTAAAATTCCCGTCGCTATC [SEQ ID NO: 5] and GACCCCCACACAGCAAA [SEQ ID NO: 4], the Tc of the COLD-PCR is 78.5° C.;
where the target is EGFR and the nucleic acids are GCAGCATGTCAAGATCACAGA [SEQ ID NO: 6] and TGCCTCCTTCTGCATGGTAT [SEQ ID NO: 7] the Tc of the COLD-PCR is 86.5° C.;
where the target is p53 and the nucleic acids are AACCAGCCCTGTCGTCTCT [SEQ ID NO: 8] and CAAGCAGTCACAGCACATGA [SEQ ID NO: 9] the Tc of the COLD-PCR is 86.7° C., optionally wherein the denaturing time is 10 seconds;
where the target is p53 and the nucleic acids are CTGAGCAGCGCTCATGGT [SEQ ID NO: 10] and GTGCAGCTGTGGGTTGATTC [SEQ ID NO: 11] the Tc of the COLD-PCR is 89° C., optionally wherein the denaturing time is 10 seconds;
where the target is p53 and the nucleic acids are GGGGGTGTGGAATCAAC [SEQ ID NO: 12] and ACTTGTGCCCTGACTTTCAA [SEQ ID NO: 13] the Tc of the COLD-PCR is 83° C., optionally wherein the denaturing time is 10 seconds;
where the target is p53 and the nucleic acids are CAGTTGCAAACCAGACCTCA [SEQ ID NO: 14] and GGCCTCTGATTCCTCACTGAT [SEQ ID NO: 15] the Tc of the COLD-PCR is 87.5° C., optionally wherein the denaturing time is 3 seconds;
where the target is p53 and the nucleic acids are GGCTCCTGACCTGGAGTCTT [SEQ ID NO: 16] and CTTGGGCCTGTGTTATCTCC [SEQ ID NO: 17] the Tc of the COLD-PCR is 83.5° C., optionally wherein the denaturing time is 10 seconds;
where the target is p53 and the nucleic acids are TCTTGCGGAGATTCTCTTCC [SEQ ID NO: 18] and GCCTCTTGCTTCTCTTTTCCT [SEQ ID NO: 19] the Tc of the COLD-PCR is 81.8° C., optionally wherein the denaturing time is 20 seconds;
where the target is p53 and the nucleic acids are GCTTCTTGTCCTGCTTGCTT [SEQ ID NO: 20] and CTACTGGGACGGAACAGCTT [SEQ ID NO: 21] the Tc of the COLD-PCR is 85.3° C., optionally wherein the denaturing time is 10 seconds;

5. The method of any of claims 2 and 3 wherein the reaction cycle is:
a)

| Stage | Temp | Time | Cycles |
|---|---|---|---|
| Activation | 95° C. | 5 min | 1 |
| Pre-PCR | 95° C. | 10 sec | 20 |
| | 55° C. | 30 sec | |
| | 72° C. | 10 sec | |
| COLD | 79° C. | 3 sec | 40/45 |
| | 55° C. | 30 sec | |
| | 72° C. | 10 sec | |
| HRM | 68-90 | | 1 |

where the target is KRAS and the reaction is fast COLD-PCR;
b)

| Stage | Temp | Time | Cycles |
|---|---|---|---|
| Activation | 95° C. | 5 min | 1 |
| Pre-PCR | 95° C. | 10 sec | 20 |
| | 60° C. | 1 min | |
| COLD | 95° C. | 10 sec | 40/45 |
| | 70° C. | 5 min | |
| | 79.5° C. | 3 sec | |
| | 55° C. | 30 sec | |
| | 72° C. | 10 sec | |
| HRM | 68-90 | | 1 |

where the target is KRAS and the reaction in full COLD-PCR;
c)

| Stage | Temp | Time | Cycles |
|---|---|---|---|
| Activation | 95° C. | 5 min | 1 |
| Pre-PCR | 95° C. | 10 sec | 20 |
| | 55° C. | 30 sec | |
| | 72° C. | 10 sec | |
| COLD | 78.5° C. | 3 sec | 40/45 |
| | 55° C. | 30 sec | |
| | 72° C. | 10 sec | |
| HRM | 68-90 | | 1 |

where the target is EGFR exon 19 and the reaction is fast COLD-PCR;
d)

| Stage | Temp | Time | Cycles |
|---|---|---|---|
| Activation | 95° C. | 5 min | 1 |
| Pre-PCR | 95° C. | 10 sec | 20 |
| | 60° C. | 1 min | |
| COLD | 95° C. | 10 sec | 40/45 |
| | 70° C. | 5 min | |
| | 86° C. | 3 sec | |
| | 55° C. | 30 sec | |
| | 72° C. | 10 sec | |
| HRM | 68-90 | | 1 |

where the target is EGFR exon 21 and the reaction is full COLD-PCR;
e)

| Stage | Temp | Time | Cycles |
|---|---|---|---|
| Activation | 95° C. | 5 min | 1 |
| Pre-PCR | 95° C. | 10 sec | 20 |
| | 55° C. | 30 sec | |
| | 72° C. | 10 sec | |
| COLD | Tc° C. | | 40/45 |
| | SEQ ID NO: 8 and 9 - 86.7° C. | 10 sec | |
| | SEQ ID NO: 10 and 11 - 89° C. | 10 sec | |
| | SEQ ID NO: 12 and 13 - 83° C. | 10 sec | |
| | SEQ ID NO: 14 and 15 - 87.5° C. | 3 sec | |
| | SEQ ID NO: 16 and 17 - 83.5° C. | 10 sec | |
| | SEQ ID NO: 18 and 19 - 81.8° C. | 20 sec | |
| | SEQ ID NO: 20 and 21 - 85.3° C. | 10 sec | |
| | 55° C. | 30 sec | |
| | 72° C. | 10 sec | |
| HRM | 68-90 | | 1 |

Where the target is p53 and the reaction is fast COLD-PCR.

6. The method of any of claims 2-5 wherein the presence of the mutation is detected by high resolution melt curve analysis or sequencing or RT-PCR.

7. The method of any of claims 2-6 wherein the presence of a mutation is used to determine appropriate treatment.

8. The method of claim 7 wherein where a cancer is determined to be positive for one or more EGFR mutations, the appropriate treatment is considered to be Gefitinib, Erlotinib or Afatanib, and/or where a cancer is determined to be positive for one or more EGFR mutations, the appropriate treatment is not considered to be panitumumab and cetuximab.

9. The method of any of claims 2-8 wherein the method further comprises administration to the subject of a suitable therapeutic.

10. The method of any of claims 2-9 further comprising the determination of the presence or absence of a mutation in one or more of the APC BRAF, ALK, PIK3CA, DDR2, HER2, FGFR1, MAP2K1, MET, NRAS, NTRK1, PTEN, RET, ROS1 genes.

11. The method of any of claims 2-10 wherein the mutation is a point mutation, deletion mutation, or insertion mutation.

12. The method of any of claims 2-11 wherein the PCR is carried out on circulating tumour DNA, optionally wherein the circulating tumour DNA is extracted from plasma.

13. The method of any of claims 2-12 wherein the sample is a tumour biopsy sample.

14. The method of any of claims 2-13 wherein the presence of more than one mutation is assessed.

15. The method of any of claims 2-14 wherein the presence of one or more mutations in more than one gene is assessed.

16. A method for characterising a tumour of a subject comprising determining the presence or absence of a mutation in the KRAS locus, according to the method of any of claims 2-15, and further comprising the determination of the presence or absence of a mutation in the APC gene.

17. The method of 16 wherein a KRAS mutation in the absence of an APC mutation is considered to indicate a high likelihood of developing a self-limiting hyperplastic or borderline lesion; and the presence of both a mutation in the APC gene and the KRAS gene is considered to indicate a high likelihood of developing cancer.

18. The method of 16 and 17 wherein where the mutation in the APC gene is known to occur prior to the mutation in the KRAS gene, the subject is deemed likely to have or develop cancer.

19. A method of predicting the likelihood of a subject developing cancer, optionally colorectal cancer, wherein the subject is assessed for mutation in both the KRAS and APC gene over a period of time.

20. A method for determining a subject's suitability for treatment with an anti-EGFR therapy, comprising assessing the presence or absence of a mutation in the EGFR and/or KRAS gene according to any of claims 2-15.

21. The method of claim 20 wherein where the subject is found to have a mutation in the EGFR gene the subject is deemed suitable for EGFR therapy.

22. The method of any of claims 20 and 21 wherein where the subject is found to have a mutation in the KRAS gene, the subject is not deemed to be suitable for anti-EGFR therapy.

23. A method of treating a subject for cancer, wherein the method comprises determination of the presence or absence of a mutation in the KRAS, p53 and/or EGFR gene according to any of claims 2-15, and further comprising the administration of a therapeutic agent if a mutation is identified, wherein the choice of therapeutic agent is determined based on the determined mutation profile.

24. A method of diagnosing a subject with cancer, or a pre-cancerous lesion, comprising the determination of the presence or absence of a mutation in the KRAS, p53 and/or EGFR gene according to any of claims 2-15, wherein the presence of a mutation indicates that the subject is likely to have, or to develop, cancer.

25. The method of claim 24 wherein the subject is assessed for other mutations or symptoms or physical presence of cancer.

26. The method of any of claims 16-25 wherein the method is performed on a sample of circulating tumour DNA, optionally wherein the circulating tumour DNA is extracted from plasma.

27. The method of any of claims 16-25 wherein the method is performed on a sample of a tumour biopsy sample.

28. Gefitinib, Erlotinib or Afatanib for use in treating cancer wherein the subject has been determined to be suitable for treatment with Gefitinib, Erlotinib or Afatanib according to the method of claim 7 or 8.

29. Use of Gefitinib, Erlotinib or Afatanib for use in the manufacture of a medicament for use in treating cancer, wherein the subject has been determined to be suitable for treatment with Gefitinib, Erlotinib or Afatanib according to the method of claim 7 or 8.

30. A kit of parts comprising any one of the nucleic acids of claim 1.

31. The kit according to claim 30 wherein the kit comprises any two of the nucleic acids of claim 1, optionally wherein the any two comprises nucleic acids with:
[SEQ ID NO: 1]
AAAACAAGATTTACCTCTATTGTTGGA
and
[SEQ ID NO: 2
AGGCCTGCTGAAAATGACTG;
[SEQ ID NO: 3]
GTTAAAATTCCCGTCGCTATCA
and
[SEQ ID NO: 4]
GACCCCCACACAGCAAA;
[SEQ ID NO: 5]
AAGTTAAAATTCCCGTCGCTATC
and
[SEQ ID NO: 4]
GACCCCCACACAGCAAA;
[SEQ ID NO: 6]
GCAGCATGTCAAGATCACAGA
and
[SEQ ID NO: 7]
TGCCTCCTTCTGCATGGTAT;
[SEQ ID NO: 8]
AACCAGCCCTGTCGTCTCT
and
[SEQ ID NO: 9]
CAAGCAGTCACAGCACATGA;
[SEQ ID NO: 10]
CTGAGCAGCGCTCATGGT
and
[SEQ ID NO: 11]
GTGCAGCTGTGGGTTGATTC;
[SEQ ID NO: 12]
GGGGGTGTGGAATCAAC
and
[SEQ ID NO: 13]
ACTTGTGCCCTGACTTTCAA;
[SEQ ID NO: 14]
CAGTTGCAAACCAGACCTCA
and
[SEQ ID NO: 15]
GGCCTCTGATTCCTCACTGAT;
[SEQ ID NO: 16]
GGCTCCTGACCTGGAGTCTT
and
[SEQ ID NO: 17]
CTTGGGCCTGTGTTATCTCC;
[SEQ ID NO: 18]
TCTTGCGGAGATTCTCTTCC
and
[SEQ ID NO: 19]
GCCTCTTGCTTCTCTTTTCCT;
[SEQ ID NO: 20]
GCTTCTTGTCCTGCTTGCTT
and
[SEQ ID NO: 21]
CTACTGGGACGGAACAGCTT.

32. The kit according to any of claims 30 and 31 wherein the kit further comprises one or more control samples, optionally wherein the control samples comprise the wild type and/or the mutant version of each PCR product generated by each pair of primers.

33. The kit according to any of claims 30-32 wherein the kit comprises standard curves generated by each mutant PCR product.

34. The kit according to any of claims 30 to 33 further comprising PCR reagents and/or detection reagents.

35. The kit according to any of claims 30-34 comprising any two of the nucleic acids of claim 1, optionally any 3, or any 4, or any 5, or any 6, or any 7, or any 8, or any 9, or any 10, or any 11, or any 12, or any 13, or any 14, or any 15, or any 16, or any 17, or any 18, or any 19, or any 20, or any 21 nucleic acids according to claim 1.

36. The kit according to any of claims 30-35 further comprising reaction wells, optionally a reaction plate, optionally a microtitre plate.

37. An anticancer agent for use in the treatment of a subject with cancer wherein the subject is assessed as being suitable for the treatment according to the method of claim 7 or 8.
